(19) United States
(12) Patent Application Publication
(10) Pub. No.: US 2024/0287455 A1
(43) Pub. Date: Aug. 29, 2024

(54) COMPUTER-BASED SYSTEMS FOR CONTROLLING AND MONITORING METABOLIC RATE AND ENVIRONMENTAL FACTORS OF AT LEAST ONE BIOREACTOR AND METHODS OF USE THEREOF

(71) Applicant: ADVA Biotechnology Ltd., Kiryat Tivon (IL)

(72) Inventors: Ohad Karnieli, Kiryat Tivon (IL); Rami Massri, Jadeida al Makr (IL); Mahmoud Abuelhija, Kaukab Abuelhija (IL); Batel Tayar, Zefat (IL); Noam Bercovich, Kiryat Byalik (IL); Helena Feldman, Halutz (IL)

(21) Appl. No.: 18/415,134

(22) Filed: Jan. 17, 2024

Related U.S. Application Data
(60) Provisional application No. 63/480,221, filed on Jan. 17, 2023.

(51) Int. Cl. C12N 5/0783; C12M 1/00; C12M 1/34; C12M 1/36

(52) U.S. Cl. CPC C12N 5/0636; C12M 29/06; C12M 41/30; C12M 41/48

(57) ABSTRACT

A bioreactor chamber for growing a cell culture in a liquid medium includes a controller, sensors coupled to the bioreactor chamber, control devices for varying a gas flow and/or a fluid flow in the bioreactor chamber. The plurality of sensors is configured to measure a plurality of sensor parameters in the liquid medium. Sensor data from each of the sensors is received at predefined time intervals. A desired cell growth configuration and the sensor data from each of the sensors are inputted into a cell culture control machine learning model. Performing, based on output data from the cell culture control machine learning model, transmitting a control circuitry command to a control device to control at least one of: a display of a cell culture parameter prediction, a gas flow, a liquid flow of a nutrient fluid, or a removal of waste products from the liquid medium.

### FIELD OF TECHNOLOGY

The present disclosure generally relates to computer-based systems, and more specifically to computer-based systems for controlling and monitoring metabolic rate and environmental factors of at least one bioreactor and methods of use thereof.

### BRIEF DESCRIPTION OF THE DRAWINGS

Various embodiments of the present disclosure can be further explained with reference to the attached drawings, wherein like structures are referred to by like numerals throughout the several views. The drawings shown are not necessarily to scale, with emphasis instead generally being placed upon illustrating the principles of the present disclosure. Therefore, specific structural and functional details disclosed herein are not to be interpreted as limiting, but merely as a representative basis for teaching one skilled in the art to variously employ one or more illustrative embodiments.

FIGS. 1A-17 show one or more schematic flow diagrams, certain computer-based architectures, and/or screenshots of various specialized graphical user interfaces which are illustrative of some exemplary aspects of at least some embodiments of the present disclosure.

### SUMMARY

In some embodiments, the present disclosure provides an exemplary technically improved computer-based method that includes at least the following steps of providing a bioreactor arrangement including: a bioreactor chamber for growing a cell culture that may include a plurality of cells or microorganisms in a liquid medium, a controller, a plurality of sensors coupled to the bioreactor chamber, a plurality of control devices for varying a gas flow, a fluid flow, or both in the bioreactor chamber, and a control circuitry may be configured for receiving control circuitry commands from the controller to control the plurality of control devices. The plurality of sensors may be configured to measure a plurality of sensor parameters in the liquid medium that may include: a dissolved oxygen (DO) level, a glucose level, and a lactate level. A desired cell growth configuration may be received by the controller through an input device. The desired cell growth configuration may include at least one desired value range, at least one setpoint value, or any combination thereof for each of the plurality of sensor parameters measured by the plurality of sensors at predefined time intervals for each of a plurality of cell culture growth parameters during the growth of the cell culture. Sensor data from each of the plurality of sensors may be received by the controller at the predefined time intervals. The desired cell growth configuration and the sensor data from each of the plurality of sensors, may be inputted into at least one cell culture control machine learning model. The at least one cell culture control machine learning model may be trained using datasets based at least in part on time-dependent correlations between the plurality of sensor parameters and a plurality of cell culture growth parameters. Based on output data from the at least one cell culture control machine learning model, the controller may perform at least one of: transmitting at least one control circuitry command to display on a display, at least one cell culture parameter prediction from the plurality of cell culture growth parameters, transmitting at least one control circuitry command to control a gas control device for varying a gas level in the bioreactor chamber, transmitting at least one control circuitry command to control a device for controlling a liquid flow of a nutrient fluid into the bioreactor chamber, or transmitting at least one control circuitry command to open a device coupled to the bioreactor chamber for removing waste products from the liquid medium.

In some embodiments, the present disclosure provides an exemplary technically improved computer-based system that includes at least the following components of a bioreactor chamber for growing a cell culture comprising a plurality of cells or microorganisms in a liquid medium; a controller; a reservoir chamber for holding a nutrient fluid including at least one nutrient for the plurality of cells or microorganisms; a plurality of sensors coupled to the bioreactor chamber, a plurality of control devices to vary a gas flow, a fluid flow, or both in the bioreactor chamber, and a control circuitry configured to receive control circuitry commands from the controller to control the plurality of control devices. The plurality of sensors may be configured to measure a plurality of sensor parameters in the liquid medium that may include: a dissolved oxygen (DO) level, a glucose level, and a lactate level. The controller may be configured to execute computer code that causes the controller to: receive, through an input device, a desired cell growth configuration; where the desired cell growth configuration may include at least one desired value range, at least one setpoint value, or any combination thereof for each of the plurality of sensor parameters measured by the plurality of sensors at predefined time intervals for each of a plurality of cell culture growth parameters during the growth of the cell culture; receive at the predefined time intervals, sensor data from each of the plurality of sensors; input the desired cell growth configuration and the sensor data from each of the plurality of sensors, into a cell culture control machine learning model; where the at least one cell culture control machine learning model may be trained using datasets based at least in part on time-dependent correlations between the plurality of sensor parameters and a plurality of cell culture growth parameters; and perform based on output data from the cell culture control machine learning model at least one of: transmit at least one control circuitry command to display at least one cell culture parameter prediction from the plurality of cell culture growth parameters on a display, transmit at least one control circuitry command to a gas control device for varying a gas level in the bioreactor chamber, transmit at least one control circuitry command to control a device for controlling a liquid flow of a nutrient fluid into the bioreactor chamber, or transmit at least one control circuitry command to open a device coupled to the bioreactor chamber for removing waste products from the liquid medium.

### DETAILED DESCRIPTION

Various detailed embodiments of the present disclosure, taken in conjunction with the accompanying figures, are disclosed herein; however, it is to be understood that the disclosed embodiments are merely illustrative. In addition, each of the examples given in connection with the various embodiments of the present disclosure is intended to be illustrative, and not restrictive.

Throughout the specification, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments may be readily combined, without departing from the scope or spirit of the present disclosure.

In addition, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

It is understood that at least one aspect/functionality of various embodiments described herein can be performed in real-time and/or dynamically. As used herein, the term “real-time” is directed to an event/action that can occur instantaneously or almost instantaneously in time when another event/action has occurred. For example, the “real-time processing,” “real-time computation,” and “real-time execution” all pertain to the performance of a computation during the actual time that the related physical process (e.g., a user interacting with an application on a mobile device) occurs, in order that results of the computation can be used in guiding the physical process.

As used herein, the term “dynamically” and term “automatically,” and their logical and/or linguistic relatives and/or derivatives, mean that certain events and/or actions can be triggered and/or occur without any human intervention. In some embodiments, events and/or actions in accordance with the present disclosure can be in real-time and/or based on a predetermined periodicity of at least one of: nanosecond, several nanoseconds, millisecond, several milliseconds, second, several seconds, minute, several minutes, hourly, several hours, daily, several days, weekly, monthly, etc.

As used herein, the term “runtime” corresponds to any behavior that is dynamically determined during an execution of a software application or at least a portion of software application.

Embodiments of the present disclosure herein disclose computer-based method and systems of a bioreactor that can be controlled by at least one cell culture control machine learning model (MLM) in real time executed by a controller. The MLM may be trained to use, in part, a plurality of sensor data output parameters generated from a plurality of sensors monitoring the bioreactor at its input and map them to output control commands that can dynamically actuate various hardware components of the bioreactor system to control the different parameters of the bioreactor, and/or predict a plurality of cell culture parameter predictions of a plurality of cells growing in a fluid medium within the bioreactor.

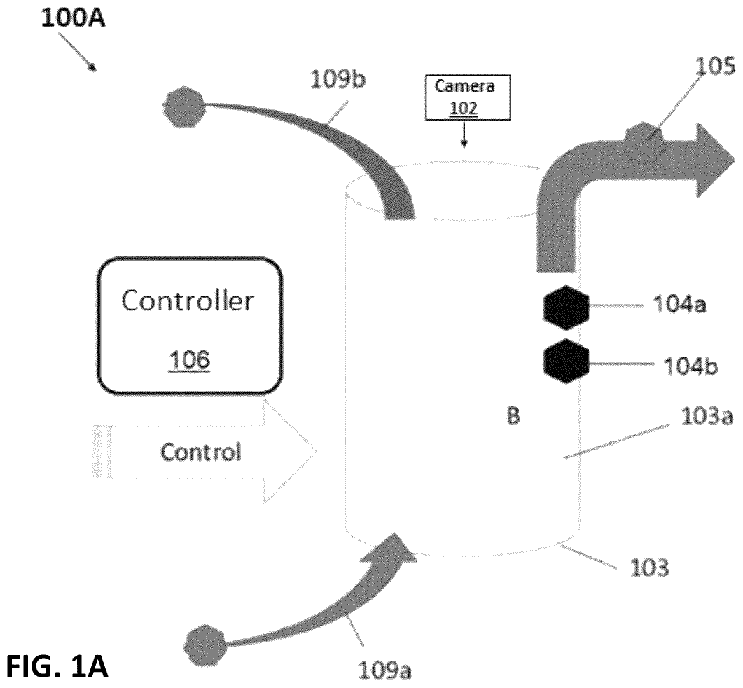

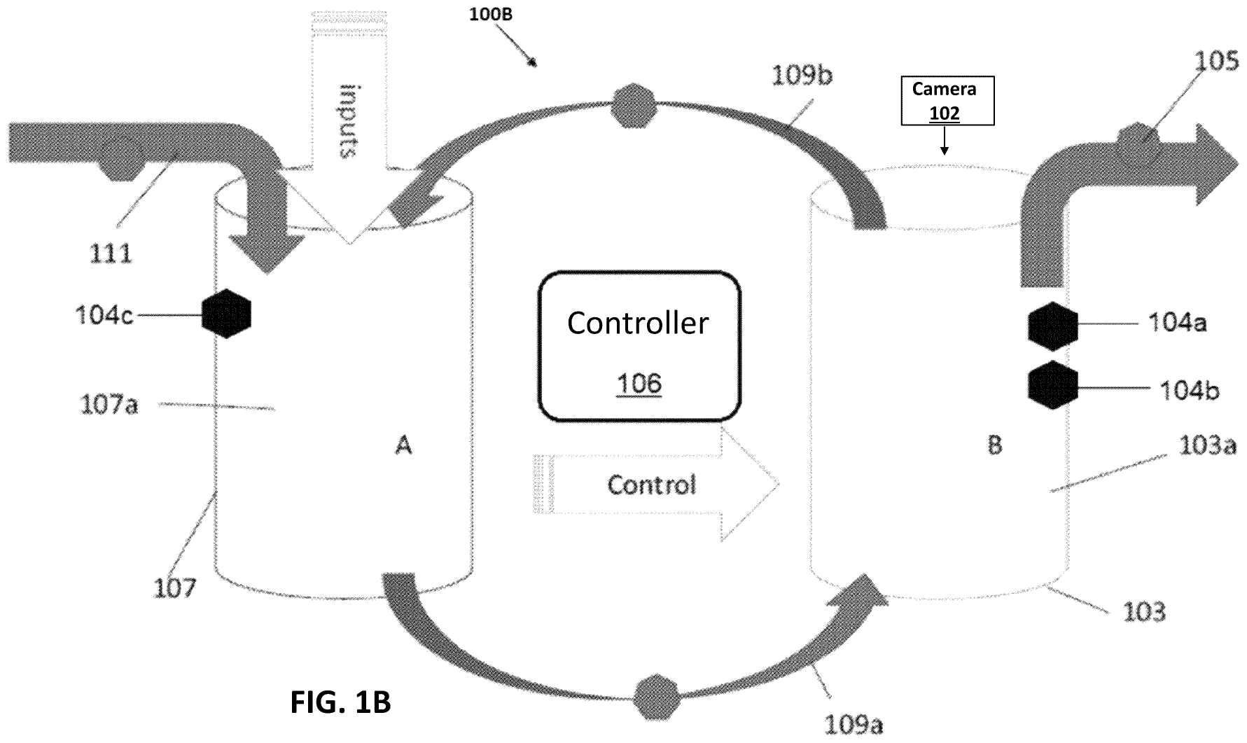

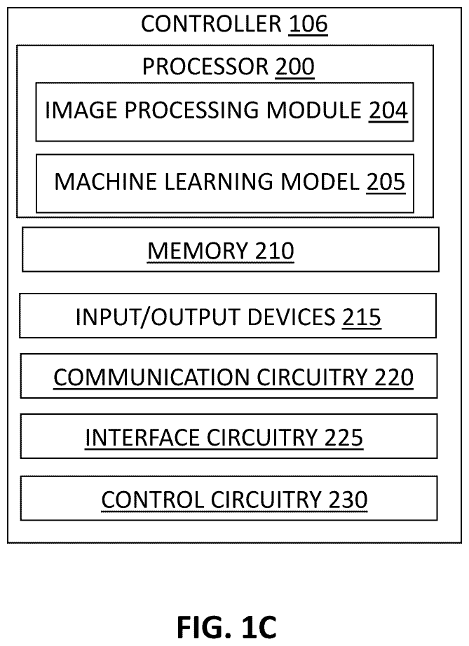

FIG. 1A is a schematic block diagram illustrating various components of a first embodiment of a bioreactor system 100A in accordance with some embodiments of the bioreactor systems of the present disclosure. In some embodiments, the exemplary bioreactor system 100A may include a bioreactor 103 which may also include an outlet 105, two pumps 109a and 109b, a plurality of sensors 104a and 104b coupled to the bioreactor 103, a controller 106, an image camera 102, and an inner chamber 103a, where the inner chamber 103a is configured to contain at least a plurality of cells.

FIG. 1B is a schematic block diagram illustrating various components of a second embodiment of a bioreactor system 100B in accordance with some embodiments of the bioreactor systems of the present disclosure. The bioreactor system 100B may include all of the elements of the bioreactor system 100A. The bioreactor system 100B may further include a reservoir chamber 107 is fluidly connected to bioreactor chamber 103 by a fluid circuit (for example, a pump system) including at least two sections (e.g., two pumps 109a and 109b). The fluid circuit of the exemplary bioreactor system includes at least two sections, each, for example, configured with a pump (109a, 109b), and the fluid circuit or the pump system generically will be referred to hereinafter as 109.

In some embodiments, the exemplary bioreactor system 100B may include the plurality of sensors 104a and 104b coupled to the bioreactor 103 and the plurality of sensors 104c coupled to the reservoir chamber 107.

In some embodiments, the reservoir chamber 107 may further include an inlet 111. In some embodiments, the reservoir chamber 107 may be another chamber with fluid medium. In some embodiments, the fluid medium in the reservoir may be the same as the fluid medium in the bioreactor. In some embodiments, the fluid medium in the reservoir may be different than the fluid medium in the bioreactor. In some embodiments, the reservoir chamber 107 does not contain any cells. In some embodiments, the reservoir is a container configured to contain a medium for providing delivery of a fluid to the bioreactor 103. In some embodiments, the reservoir is a container configured to receive and contain waste from the bioreactor 103. In some embodiments, the bioreactor system 100B includes at least two reservoirs, a first reservoir chamber 107 configured to contain a medium for providing fluid to the bioreactor 103, wherein the fluid is received via the inlet 111; and a second reservoir (not shown in FIG. 1A) configured to receive and contain waste from the bioreactor 103, via a fluid circuit (not shown in FIG. 1A) or via the outlet 105.

In some embodiments, the bioreactor 103 may include a first fluid medium. In some embodiments, the first fluid medium may include at least one gas, at least one nutrient, where the at least one nutrient may be present in a sufficient amount so as to feed the plurality of cells, a liquid, or any combination thereof. The liquid of the first fluid medium may further include a volume of the liquid in the bioreactor 103.

In some embodiments, the liquid may have a temperature ranging from 37 Celsius to 42 Celsius. In some embodiments, the liquid may have a temperature ranging from 24 Celsius to 42 Celsius. In some embodiments, the liquid may have a temperature ranging from 2 Celsius to 70 Celsius. In some embodiments, the liquid may have a pH level ranging from 6.5 pH to 7.5 pH. In some embodiments, the liquid may have a pH level ranging from 5 pH to 8 pH.

In some embodiments of the exemplary bioreactor system 100, the reservoir may have an inner chamber 107a configured to contain at least a second fluid medium. The second fluid medium may include at least one gas, at least one nutrient in a sufficient amount to feed the at least a plurality of cells, a liquid, or any combination thereof. The liquid of the second medium may further include a volume of the liquid in the reservoir chamber 107, a temperature and a pH level. Nutrients which may be used for culturing in an exemplary bioreactor system may include, but are not limited to, glucose, lactate, glutamine, glutamate, or a combination thereof. One or more gases that may be used for culturing in an exemplary bioreactor system may include, but are not limited to, oxygen, nitrogen, carbon dioxide, air, or any combination thereof. In some embodiments, the one or more gases are dissolved gases (e.g., dissolved in the medium).

The bioreactor 103, in some embodiments, may further include at least two sensors (not shown) configured to measure a plurality of parameters both physical and chemical in a fluid medium and cells contained in the bioreactor. In some embodiments, the bioreactor 103 may further include at least three sensors. In some embodiments, the bioreactor 103 may further include at least four sensors. In some embodiments, the bioreactor may include five or more sensors. In some embodiments, the reservoir chamber 107 of the exemplary bioreactor system may contain at least one sensor configured to measure parameters both physical and chemical in a fluid medium contained in the reservoir. In some embodiments, the reservoir chamber 107 may further include at least 2 sensors. In some embodiments, the reservoir chamber 107 may further include at least 3 sensors. In some embodiments, the reservoir chamber 107 may further include at least 4 sensors. In some embodiments, the reservoir chamber 107 may further include 5 or more sensors.

In some embodiments, the parameters sensed and measured in an exemplary bioreactor system (e.g., via sensors) may be selected from at least, but not limited to: a level of cell concentration; a level of the at least one nutrient; a level of at least one gas; a volume of liquid of the first medium; a pH level of a liquid of the first medium; a temperature of a liquid of the first medium; or any combination thereof.

In some embodiments, the parameters are sensed, detected, measured, controlled, or any combination thereof. In some embodiments of an exemplary bioreactor system, these parameters are selected from at least, but not limited to: a level of cell concentration contained in a bioreactor chamber; a rate of flow of a fluid into a reservoir; a rate of flow of the same or a different fluid into the bioreactor chamber; a volume of at least one fluid; a pH of at least one fluid; a temperature of at least one fluid; a level of dissolved oxygen of at least one fluid; a level of dissolved CO2 in at least one fluid; a level of HCO₃ in at least one fluid; a level of nutrient in at least one fluid; and any combination thereof.

In some embodiments, the parameters sensed and measured may be, but are not limited to, a temperature, a pH level, a glucose concentration, dissolved oxygen concentration, lactate concentration, glutamine concentration, glutamate concentration, a concentration of dissolved carbon dioxide, a concentration of HCO₃ ions, and any combination thereof.

In some embodiments, at least three of the above parameters are detected by sensors of the bioreactor system. In some embodiments, at least three of the above parameters are measured by the bioreactor system. In some embodiments, at least three of the above parameters are controllable by the configuration of the bioreactor system (e.g., via a control device configured to control a fluid circuit). In some embodiments, an exemplary bioreactor system may control the parameters sensed and measured to a predetermined set point or a predetermined range of that measurement. In some embodiments, the exemplary bioreactor system may control 1 to 5 parameters. In some embodiments, the exemplary bioreactor may control 5 to 10 parameters. In some embodiments, the exemplary bioreactor may control at least three parameters simultaneously, substantially simultaneously, or the input of the control of multiple parameters is not simultaneous but the activation of the fluid circuit in the bioreactor system based on the input of the control does affect changes to these parameters simultaneously.

Returning to FIG. 1A, the fluid circuit may include a fluid circuit 109 may be configured such that a first pump 109a extends from the reservoir chamber 107 into the bioreactor 103; and a second pump 109b, extends into the at least one reservoir from the at least one bioreactor. In some embodiments, inlet 111 of reservoir chamber 107 may be used to input materials for bioreactor cultures. In some embodiments outlet 105 of reservoir chamber 107 may be used to remove spent waste, medium, or any combination thereof.

In some embodiments, this configuration of the fluid circuit (e.g., the pump system) 109, in an exemplary bioreactor system, results in the reservoir chamber 107 controlling all of the inputs to the bioreactor via the controller 106 or component thereto, such as a processor. In some embodiments, the reservoir chamber 107 may control the parameters of the bioreaction or cells and a first fluid medium in the bioreactor using the unique pump system in conjunction with a reservoir.

In some embodiments, the exemplary bioreactor system measures at least one parameter in the bioreactor and with that measurement may control that parameter by making changes to parameters in the reservoir. In some embodiments of the exemplary bioreactor system, the measurements of parameters are made in the bioreactor 103 and are controlled by making changes to parameters in the reservoir chamber 107. In some embodiments, the exemplary bioreactor system may make changes to parameter or parameters in the reservoir to affect changes in the parameters in the bioreactor 103 by using the pump system 109. In some embodiments of the exemplary bioreactor system, the measurements of parameters are made in the bioreactor 103 and are only controlled by making changes to parameters in the reservoir chamber 107. For example, in some embodiments, the pH level of the bioreactor first medium may be controlled to be at a range of 6.5 pH to 7.5 pH, by controlling the range of the reservoir fluid medium in a range of 5 pH to 8 pH while controlling other set points. In some embodiments, the following parameters, introduced by pump system 109 may also be measured by sensors in either the bioreactor or the reservoir: a rate of flow of liquid of the bioreactor into the reservoir, a rate of flow of liquid from the reservoir into the bioreactor, or any combination thereof.

In some embodiments, an exemplary bioreactor system may require more medium and nutrients and larger culturing volumes. In some embodiments, the exemplary bioreactor system may be configured to allow the volume of the liquid medium in the bioreactor to be varied and allow additional medium to be added without the need to transfer the cells to a separate container.

In some embodiments, suitable control devices for controlling a gas and/or fluid flow may include, but are not limited to, one or more valves and/or one or more pumps, for example, as shown herein.

In some embodiments, the exemplary bioreactor system may adjust volume of medium contained in the bioreactor with the pump system.

In some embodiments, the pump system may be configured to remove at least some of the liquid from the bioreactor, to add at least some of the reservoir liquid to bioreactor liquid such that the volume of the bioreactor liquid is adjustable, or any combination thereof.

In some embodiments, the exemplary bioreactor system 100B may alternate between culturing modes, as detailed below. In some embodiments, alternating between the culturing modes may facilitate the ability of the bioreactor system 100B to control a plurality of parameters simultaneously.

In some embodiments, the system includes a batch culturing mode. In some embodiments, the exemplary bioreactor system may process in a batch culturing mode by the reservoir chamber 107 accepting the predetermined amount of medium and pumping the medium to the bioreactor. The bioreactor then may release waste product through outlet 105.

In some embodiments, the system includes a fed batch culturing mode. In some embodiments, the exemplary bioreactor system may process a fed batch mode by the reservoir chamber 107 accepting the predetermined amount of medium and pumping this medium to the bioreactor. In some embodiments, the process is then repeated for a predetermined amount of time. In some embodiments, the predetermined amount of time is 1 day to two months. In some embodiments the predetermined amount of time is 2 days to 4 months.

In some embodiments, the system includes a perfusion culture mode. In some embodiments, the exemplary bioreactor system may process a perfusion culture mode by the reservoir chamber 107 accepting through inlet 111 medium and pumping this same medium to the bioreactor. Sensors detect parameters so that only equivalent waste product is removed though outlet 105 of the bioreactor.

In some embodiments, the system includes a recirculation mode. In some embodiments, the exemplary bioreactor system may process a recirculation culture mode by a first pump 109a continually pumping the liquid medium from the reservoir to the bioreactor and a second pump 109b continually pumping the liquid medium from the bioreactor to the reservoir.

FIG. 1C is a block diagram of the controller 106 controlling the bioreactor systems 100A and 100B in accordance with one or more embodiments of the present disclosure. The controller 106 may include a processor 200, a non-transitory memory 210, input and/or output devices 215, communication circuitry 220, interface circuitry 225, and/or control circuitry 230. The processor 200 may be configured to execute the cell culture control machine learning model 205 and an image processing module 204. The processor 200 may be configured to receive sensor data from the plurality of sensors 104a, 104b, 104c via the interface circuitry 225 and to generate commands for digitally controlling devices via the control circuitry 230.

In some embodiments, the plurality of sensors may also include at least one imaging sensor 102 shown on FIG. 1A of any suitable type (e.g., an imaging camera) that may be placed at any position in and/or around the bioreactor chamber and/or the reservoir to output image data of an image any portion of the bioreactor including generating images of the cell culture growing in the bioreactor for analysis by the controller 106. The images of the cell culture via the interface circuitry 225 may be analyzed and processed by the image processing module 204.

In some embodiments, an automatic cell counter (not shown) may be configured to automatically microsample the cell culture by any suitable method to determine a cell count within the cell culture. The at least one imaging camera may be mounted on a microscope imaging the cells in the microsample. The images may be analyzed in the image processing module 204 so as to determine the types and number of different cells in the cell culture at any times either set by an operator or by control of the cell culture control machine learning model 205.

In some embodiments, a cell analyzer may be used to test activity and/or cell phenotype within the cell culture in the bioreactor chamber.

In some embodiments, the input/output devices 215 of the controller 106 may be configured to include any display device known in the art for displaying processed results and the values of any sensed parameters to an operator or user of the system 100. The controller 106 may be configured to also include one or more user interface device (such as, but not limited to a mouse, a light pen, a pointing device, a keyboard, a touch sensitive screen, or any other input device known in the art) which is configured to be used by the user or operator of the bioreactor systems 100A and 100B for inputting data and/or suitable commands into the controller 106. For example, the user may control the bioreactor 103 by entering suitable commands via the one or more user interface device into the controller 106 resulting in suitable control signals being sent by the controller 106 via the control circuitry 230 to any of the bioreactor fluid control devices. The user may enter any suitable parameters via the one or more user interface devices as inputs to the MLM 205 and to view on the display any outputs generated from the MLM 205.

In some embodiments, computer-controlled sensors and/or electronically controlled components of a bioreactor system that may be monitored and/or controlled via the interface circuitry 225 for sampling the plurality of sensors and/or via the control circuitry 230 may include, for example, but are not limited to:
(1) An agitator or stirrer for keeping the contents of the bioreactor mixed, ensuring proper distribution of nutrients, oxygen, and heat. Different types of impellers (paddles, turbines) may be used depending on the specific needs of the culture.
(2) A Temperature Control System for maintaining an optimal temperature for cell growth and activity. Bioreactors may use heating jackets, internal coils, and/or even water baths to achieve this.
(3) A pH Control System that may use any suitable pH sensors and/or dosing systems to adjust the acidity or alkalinity of the culture medium to keep it within the desired pH ranges.
(4) Aeration and Gas Supply to provide oxygen to the culture. Some bioreactors may use spargers to introduce bubbles, while others may rely on agitation itself or membrane technology. Other gases like CO2 may also be controlled.
(5) Nutrient Inlet-Outlet to provide fresh nutrients to the culture that may be fed into the bioreactor, and waste products may be removed. This may be performed through computer-controlled ports and/or integrated tubes.
(6) Sensors and/or Analytical Instruments for monitoring key parameters such as for example but not limited to oxygen levels, pH, and/or cell density. The bioreactor 103 may include any suitable various sensors and/or probes coupled thereon so as to provide real-time data.
(7) Computer-controlled sampling Ports for taking samples of the culture for analysis without disrupting the process.
(8) Sterilization System for keeping the bioreactor clean and free of contamination. Steam sterilization, for example, may be used. Presterilized single use systems may also be used.
(9) Computer-controlled Sparger and/or Air Inlet for introducing gas like air or oxygen directly into the culture medium through the sparger and/or diffuser.
(10) Computer-controlled Foam System for managing excessive foaming that may disrupt mixing and oxygen transfer. Anti-foaming agents and/or mechanical devices may be deployed by the computer-controlled foam system to manage this.

The embodiments shown in FIG. 1A show the basic components of a bioreactor system merely for conceptual clarity in illustrating how a machine learning model may monitor and/or control cell growth by processing sensor data and/or controlling control devices of the bioreactor system. The bioreactor 103 may include any suitable form factor and any suitable number of additional elements (not shown in FIG. 1A) to provide for optimal and adaptive culturing, such as for example, and not limited to a bioreactor system 100A and 100B elements shown in FIGS. 1A to 1C. For example, different embodiments of the bioreactor may be described hereinbelow and in the following references. More detailed embodiments of bioreactor systems may be found, for example, in the following U.S. Patents and Patent Applications, which are incorporated herein in their entirety: U.S. Pat. No. 11,549,090 filed Aug. 21, 2017, U.S. Pat. No. 11,667,882 filed Mar. 7, 2022, U.S. Pat. No. 11,859,163 filed Oct. 24, 2022, and U.S. patent application Ser. No. 17/562,586 filed Dec. 27, 2021.

In some embodiments, the bioreactor 103 may include a chamber with a widening shape, for example a conical frustum shape, or a portion thereof, which is configured to lead to reduction of velocity of a fluid. Thus, in this case, the bioreactor may be referred to herein as a “cone” or “bioreactor cone”.

In some embodiments, the bioreactor 103 may include a chamber of two parts divided by a perforated barrier, where the barrier allows a constant fluid flow, for example but not limited to a fluid growth media, and where the plurality of cells may be retained in the second (upper) chamber. Any of the plurality of sensors denoted 104a and 104b may be coupled to both a cell growth sub-chamber (cone) and/or a media (second) sub-chamber to sample any of the sensor parameters.

A skilled artisan would appreciate that the term “perforated barrier” may be used interchangeably with the term “filter” or “membrane” or “perforated plate” having all the same qualities and meanings.

In some embodiments, the perforated barrier may include a plurality of perforations therein that is configured to allow bidirectional flow of a liquid, for example a growth media through the perforations of the perforated barrier such that liquid can flow from the first chamber to the second chamber and also from the second chamber to the first chamber.

A skilled artisan would appreciate that the term “first chamber” as used herein, may in some embodiments be used interchangeably with the term “lower chamber” having all the same meanings and qualities thereof. A skilled artisan would appreciate that the term “second chamber” as used herein, may in some embodiments be used interchangeably with the term “upper chamber” having all the same meanings and qualities thereof. In some embodiments, cells are cultured in the second chamber of bioreactor vessel.

In some embodiments, the cell types compatible with growth in the bioreactor 103 disclosed herein may include, but are not limited to stem cells, Acinar cells, Adipocytes, Alveolar cells, Ameloblasts, Annulus Fibrosus Cells, Arachnoidal cells, Astrocytes, Blastoderms, Calvarial Cells, Cancerous cells (Adenocarcinomas, Fibrosarcomas, Glioblastomas, Hepatomas, Melanomas, Myeloid Leukemias, Neuroblastomas, Osteosarcomas, Sarcomas) Cardiomyocytes, Chondrocytes, Chordoma Cells, Chromaffin Cells, Cumulus Cells, Endothelial cells, Endothelial-like cells, Ensheathing cells, Epithelial cells, Fibroblasts, Fibroblast-like cells, Germ cells, Hepatocytes, Hybridomas, Insulin producing cells, Intersticial Cells, Islets, Keratinocytes, Lymphocytic cells, Macrophages, Mast cells, Melanocytes, Meniscus Cells, Mesangial cells, Mesenchymal Precursor Cells, Monocytes, Mononuclear Cells, Myeloblasts, Myoblasts, Myofibroblasts, Neuronal cells, Nucleus cells, Odontoblasts, Oocytes, Osteoblasts, Osteoblast-like cells, Osteoclasts, Osteoclast precursor cells, Oval Cells, Papilla cells, Parenchymal cells, Pericytes, Peridontal Ligament Cells, Periosteal cells, Platelets, Pneumocytes, Preadipocytes, Proepicardium cells, Renal cells, Salisphere cells, Schwann cells, Secretory cells, Smooth Muscle cells, Sperm cells, Stellate Cells, Stem Cells, Stem Cell-like cells, Stertoli Cells, Stromal cells, Synovial cells, Synoviocytes, T Cells, Tenocytes, T-lymphoblasts, Trophoblasts, Natural killer cells, dendritic cells, Urothelial cells, Vitreous cells, and the like; the cells originating from, for example and without limitation, any of the following tissues: Adipose Tissue, Adrenal gland, Amniotic fluid, Amniotic sac, Aorta, Artery (Carotid, Coronary, Pulmonary), Bile Duct, Bladder, Blood, Bone, Bone Marrow, Brain (including Cerebral Cortex), Breast, Bronchi, Cartilage, Cervix, Chorionic Villi, Colon, Conjunctiva, Connective Tissue, Cornea, Dental Pulp, Duodenum, Dura Mater, Ear, Endometriotic cyst, Endometrium, Esophagus, Eye, Foreskin, Gallbladder, Ganglia, Gingiva, Head/Neck, Heart, Heart Valve, Hippocampus, Iliac, Intervertebral Disc, Joint, Jugular vein, Kidney, Knee, Lacrimal Gland, Ligament, Liver, Lung, Lymph node, Mammary gland, Mandible, Meninges, Mesoderm, Microvasculature, Mucosa, Muscle-derived (MD), Myeloid Leukemia, Myeloma, Nasal, Nasopharyngeal, Nerve, Nucleus Pulposus, Oral Mucosa, Ovary, Pancreas, Parotid Gland, Penis, Placenta, Prostate, Renal, Respiratory Tract, Retina, Salivary Gland, Saphenous Vein, Sciatic Nerve, Skeletal Muscle, Skin, Small Intestine, Sphincter, Spine, Spleen, Stomach, Synovium, Teeth, Tendon, Testes, Thyroid, Tonsil, Trachea, Umbilical Artery, Umbilical Cord, Umbilical Cord Blood, Umbilical Cord Vein, Umbilical Cord (Wartons Jelly), Urinary tract, Uterus, Vasculature, Ventricle, Vocal folds and cells, or any combination thereof. In some embodiments, the cells grown in a bioreactor disclosed herein may comprise a combination of different cell types. As used herein, in some embodiments the terms “cells” and “microorganisms” may be used interchangeably having all the same meanings and qualities.

In some embodiments, the bioreactor 103 may be configured to include one or more sensors 104a, 104b, and 104c that may be suitably coupled and/or connected to the controller 106 via the interface circuitry 225 for monitoring and/or regulating various physical and/or chemical parameters within the growth medium (such as, for example, temperature, pH, glucose concentration, dissolved oxygen concentration the concentration of dissolved carbon dioxide or of HCO₃-ions, the concentration of lactate, and ionic strength) in the growth medium, all of which can be sensed, monitored, and controlled in the bioreactor and/or bioreactor headspace and/or in a fluid reservoir connectable to the bioreactor and/or at the various inlets or outlet ports. In some embodiments, sensors are configured to detect a product synthesized by a cell or microorganism grown in the bioreactor. In some embodiments, control of some of the features above may require mixing of the growth medium, the mixing can be provided at the fluid reservoir.

In some embodiments, the computer-based systems of the bioreactor of the present disclosure controls and monitors metabolic and environmental factors such as Oxygen, pH, Glucose, Lactate, Glutamine, Glutamate. For example, the computer-based systems of the bioreactor of the present disclosure are applied to different biological materials such as cells that consume or produce different rates of O2 for example depended on their state. Additionally, the computer-based systems of the bioreactor of the present disclosure may be applied to different type of cells consumes or produce differently.

In some embodiments, the computer-based systems of the bioreactor of the present disclosure may be applied to non-activated T cells that may consume a low amount of O2 and Glucose and may produce a very low amount of Lactate. Subsequently, once T cells are activated, the consumption rate of all the above changes as they start not only to proliferate but change their metabolism and move from energy cycle of O2 to Lactate, meaning more lactate secretion per cell. Such activity depends on the amount of activation/stimulation level which is achieved via addition of antibodies to the media. Within a few days and depending on stimulation levels, the T cells may slow down and may return to the natural state or become exhausted (less Lac production per cell). In some embodiments, the computer-based systems of the bioreactor of the present disclosure measure and control, for example, bacterial contamination, the rate of O2 consumption and of Glucose as these increase dramatically.

In some embodiments, the computer-based systems of the bioreactor of the present disclosure are applied to T cells when the T cells are introduced to their targets. This target will activate the T cell which in turn will change the consumption and production rates.

In some embodiments, the computer-based systems of the bioreactor of the present disclosure applies the online monitoring to identify such changes and predict the state of the cells in terms of at least one of the following activation, transduction, contamination in the culture, differentiation, cell death, exhaustion and/or even phenotype and killing capability.

In some embodiments, the computer-based systems of the bioreactor of the present disclosure are applied to primary cells, such as for example, but not limited to mesenchymal stem cells that change metabolism once they are stimulated to proliferate until they are inhibited by contact inhibition once the surface is covered (confluence). Subsequently, once these primary cells are activated, the consumption rate of all the above changes as they start not only to proliferate but change their metabolism and move from energy cycle of O2 to Lactate, meaning more lactate secretion per cell. Such activity depends on the amount of activation/stimulation level which is achieved via addition of antibodies to the media. Within a few days and depending on stimulation levels, these primary cells slow down and return to the natural state or become exhausted (less Lac production per cell). In some embodiments, the computer-based systems of the bioreactor of the present disclosure measure and control, for example, bacterial contamination, the rate of O2 consumption and of Glucose as these increase dramatically.

In some embodiments, the computer-based systems of the bioreactor of the present disclosure may be applied to these primary cells when the cells are introduced to their targets. This target will activate these primary cells which in turn will change the consumption and production rates.

In some embodiments, the computer-based systems of the bioreactor of the present disclosure may apply online monitoring to identify such changes and predict the state of the cells in terms of at least one of the following activation, transduction, contamination in the culture, differentiation, cell death, exhaustion and/or even phenotype and killing capability.

In some embodiments, a culture chamber (e.g., an assay) may include the same plurality of sensors 104a, 104b, and 104c as in bioreactor systems 100A and 100B above. A plurality of cells may be taken from the bioreactor 103 and may be introduced into the culture chamber to measure the state of the cells and be monitored by the cell culture control machine learning model 205.

In some embodiments, the computer-based systems of the bioreactor of the present disclosure may be applied to cells (e.g. stem cells) where cells grow until they are stimulated to differentiate and then identify when the cells differentiate—the consumption rate of all the above changes as they start not only to proliferate, but also change their metabolism and move from energy cycle of O2 to Lactate, meaning more lactate secretion per cell. In some embodiments, the computer-based systems of the bioreactor of the present disclosure measure and control, for example, bacterial contamination, the rate of O2 consumption and of Glucose as these increase dramatically.

In some embodiments, the computer-based systems of the bioreactor of the present disclosure may be applied as an online QC to insure sterility. In some embodiments, the computer-based systems of the bioreactor of the present disclosure may be applied to move to a different stage of the culture or intervene during the culture to reactivate the cells or predict when they will be ready.

In some embodiments, the computer-based systems of the bioreactor of the present disclosure may be applied so that not only do the cells change the metabolism but to measure and control the “time lapse” (e.g., parametric changes over time) and/or slopes and/or the differences (e.g., delta) between parameter values points to predict outcomes.

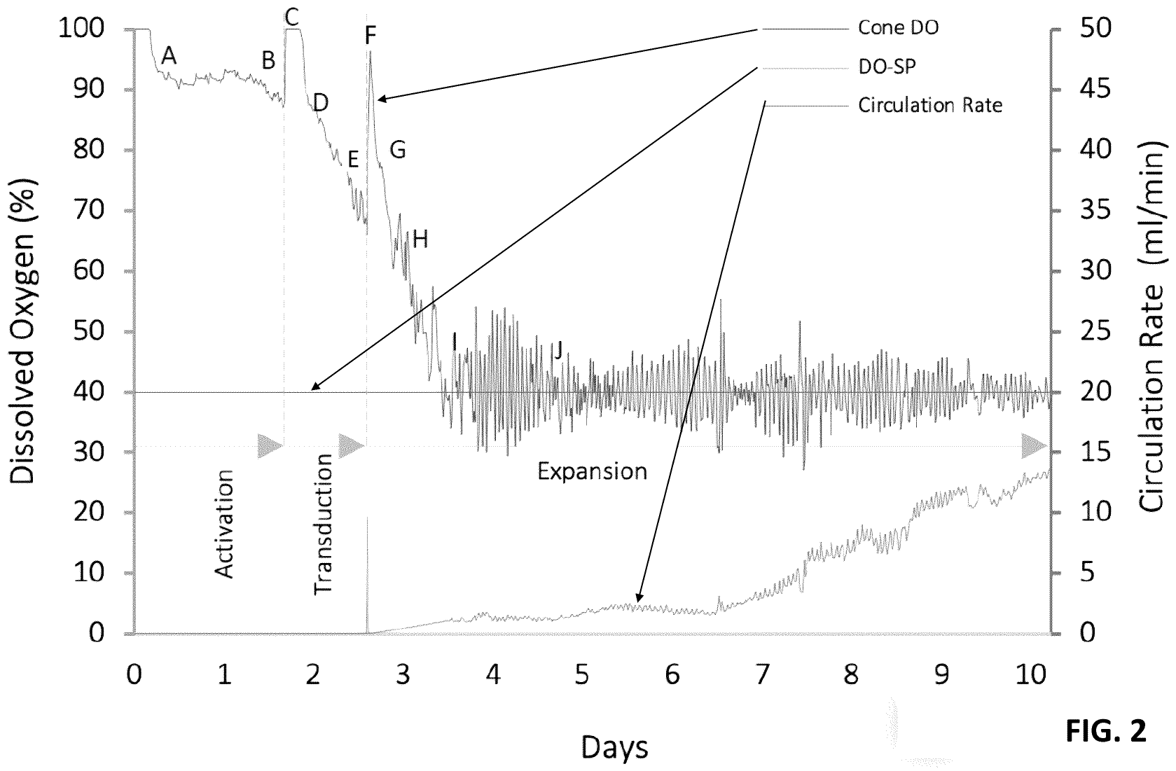

FIG. 2 is sensor measurement output data from the bioreactor in accordance with one or more embodiments of the present disclosure. The top trace is the O2 level in the cone. The bottom trace is a circulation rate of the pump feeding the cone. The marker on 40% is the setpoint (SP) that activates perfusion. Points A-B are non-activated cells showing a steady Oxygen consumption. Point C is the time where the activation reagents may be added. Points D-E may be where the cells consume significantly more Oxygen. Between Points E and G is where the virus is introduced for genetic manipulation. Points I to J is where the pump starts working. The processor 200 may calculate the slope here. In other embodiments, the pump may be stopped for X min and the slope can again be measured, where X is any suitable positive number.

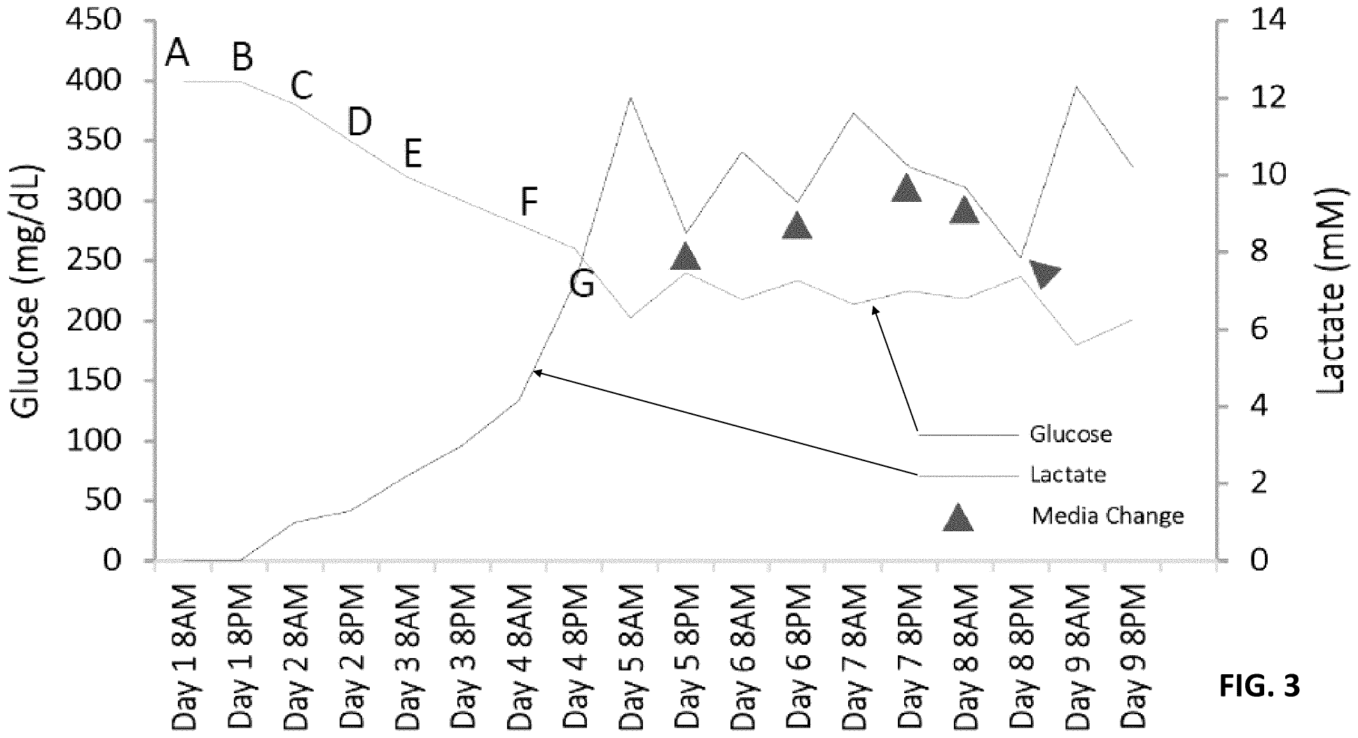

FIG. 3 is a graph of glucose (Glu) and Lactate (Lac) levels taken from the same data of FIG. 2 (e.g., the same run). However, the slope of the Glu changes differently than the Lac. In some embodiments, the computer-based systems of the bioreactor of the present disclosure may be applied so that the result may correlate the difference (e.g., delta) between the slopes of one parameter to the same parameter at a different time point, and/or the changes between the differences (delta's) of different parameters. As such, time lapse concept may be used to review and compare the change in the consumption over an X amount of time compared to the slope in later stages, where X is any suitable positive number.

In some embodiments, the processor 200 evaluating a slope in A/B where A=GLU and B=LAC may almost flat, but once the cells in the cell culture may be activated the slope of A/B may be very steep.

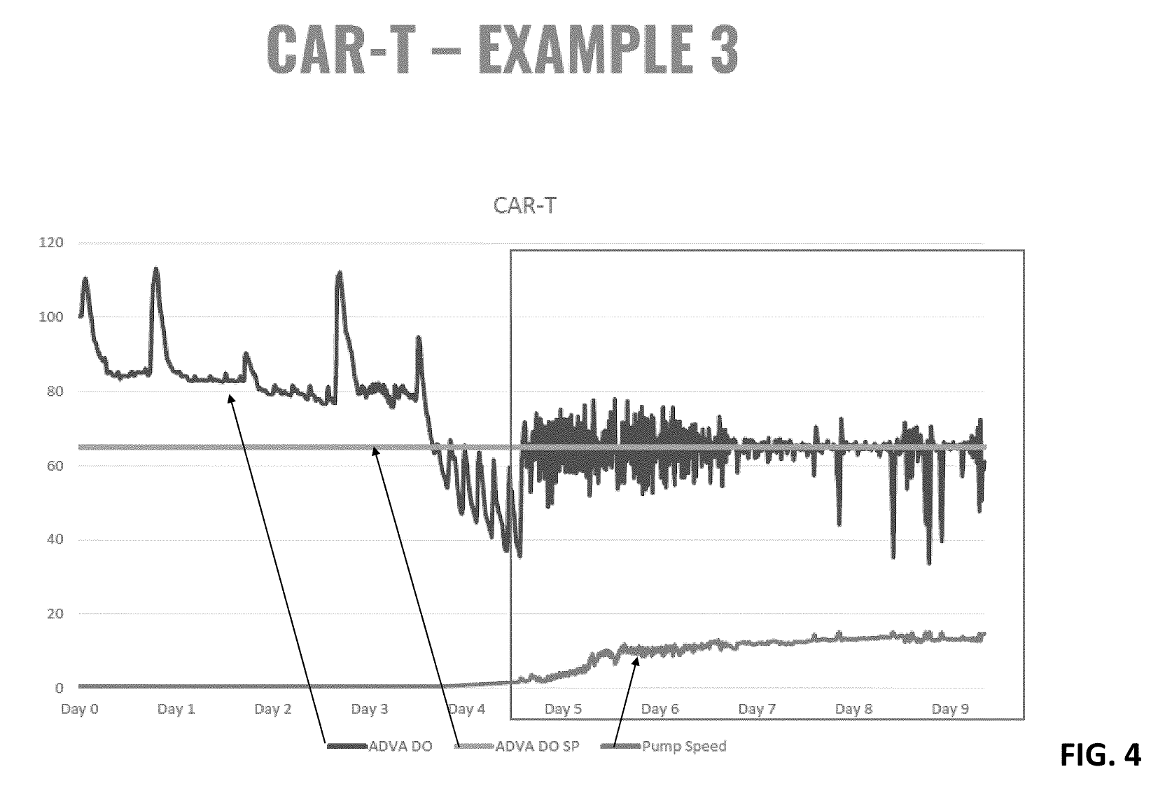

In some embodiments, the computer-based systems of the bioreactor of the present disclosure may be applied as illustrated in FIG. 4. FIG. 4 shows that the pump rate of O₂ increasing correlates to a dissolved O₂ (DO) increase in the cell culture, and thus the cells may still be activated and growing.

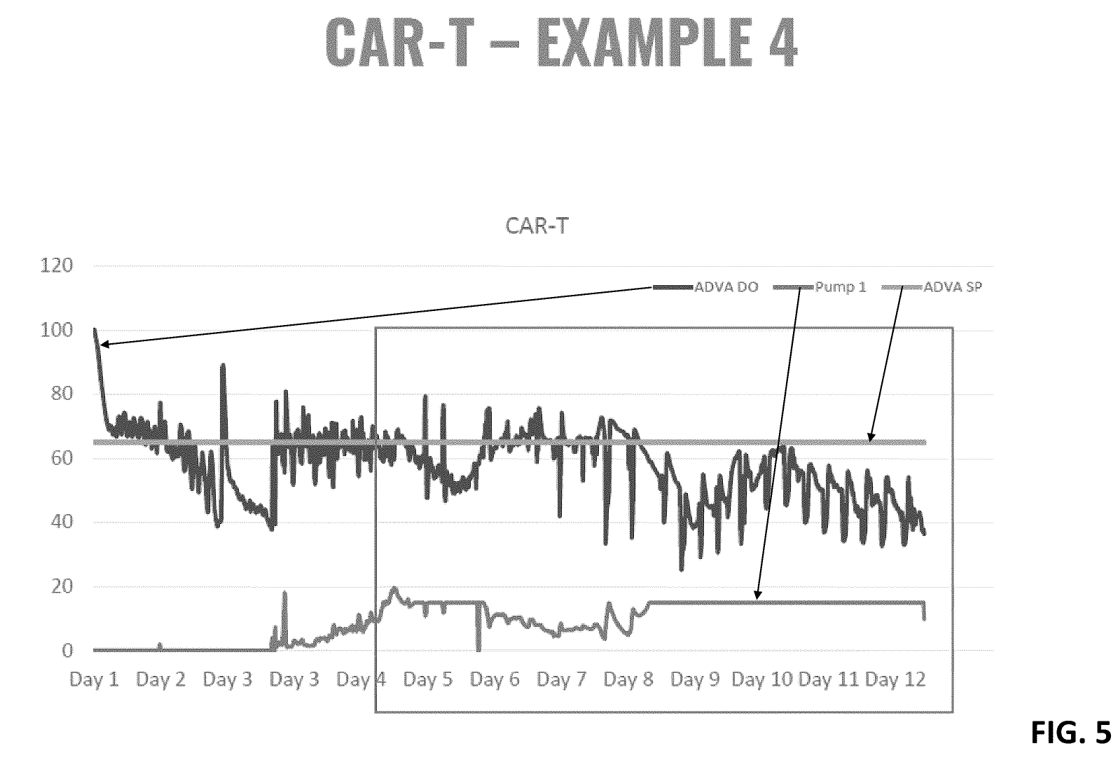

In some embodiments, the computer-based systems of the bioreactor of the present disclosure are applied as illustrated in FIG. 5. FIG. 5 is a graph of dissolved oxygen (DO) level and O2 pump rate both versus time. The graph also shows the DO set point value. Here, inside the box, the pump is at a first maximum event at day 5 and a second maximum event again at day 8 where the cells consume more O2 and then less and once again switch at day 9. The cells change their metabolism that correlates with a change in the phenotype or in the cell population that is growing. As such, the changes in the consumption/secretion of factors and metabolic factors are used to correlate the state and/or change culture conditions. Stated differently, this metabolic change correlates to the phenotype change in the cell state or a phenotypic protein change in the cell, binding the change to phenotype and activity and then to prediction of number and state.

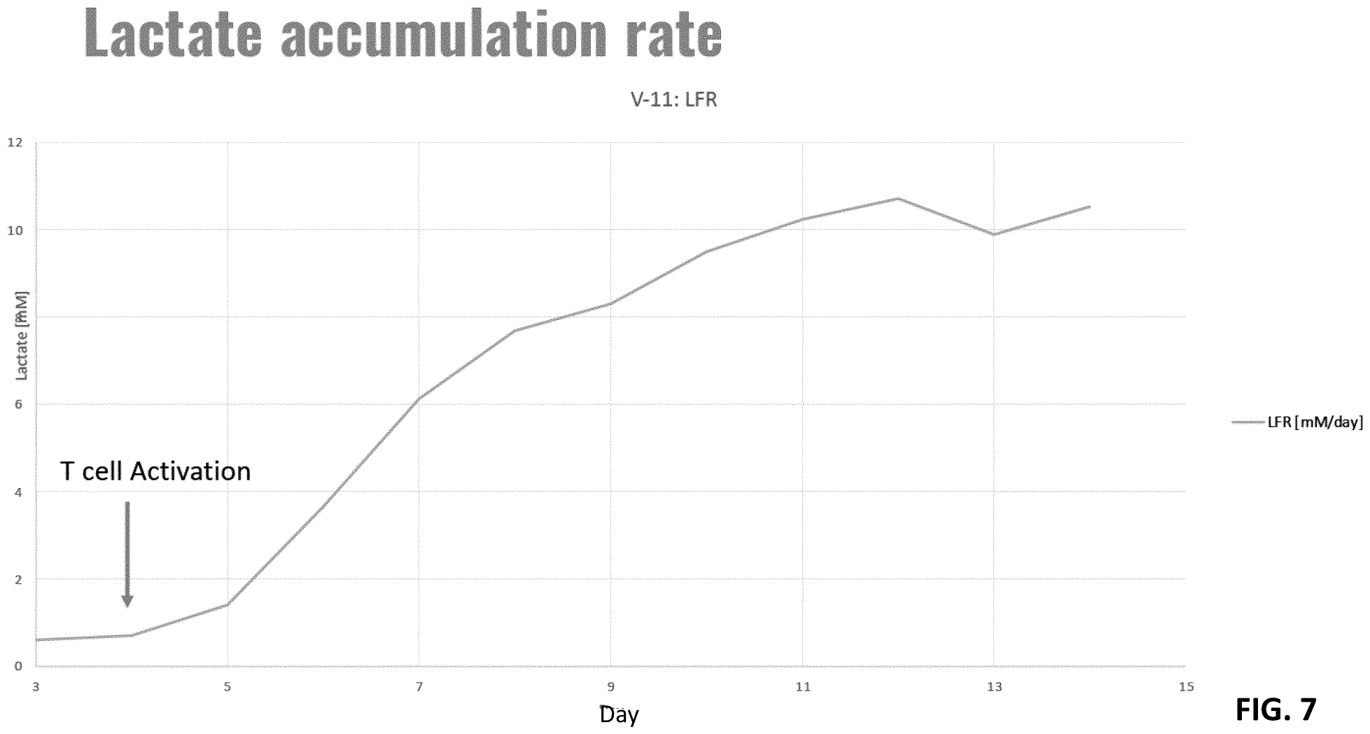

In some embodiments, the computer-based systems of the bioreactor of the present disclosure are applied as illustrated in FIG. 7. FIG. 7 illustrates the changes in lactate level as a function of time (days) in an exemplary run of Lac.

In some embodiments, the computer-based systems of the bioreactor of the present disclosure may be used to correlate between multiparameter changes/slopes (O2, pH, Glu, Lac levels) at different time points so as to predict the state of the culture and be used for several different applications.

In some embodiments, the computer-based systems of the bioreactor of the present disclosure may apply a graph of a bacterial contaminated run as supportive data and based on this data correlate desired outcomes.

In some embodiments, the computer-based systems of the bioreactor of the present disclosure may apply supportive data to result in lower and/or higher activation rates or percentage of activated cells.

In some embodiments, the computer-based systems of the bioreactor of the present disclosure may apply supportive data to show the effect of adding activation reagents once the cells slow down increasing the proliferation.

In some embodiments, the computer-based systems of the bioreactor of the present disclosure may apply supportive data of samples of cells in the different stages and determine a correlation to phenotype.

In some embodiments, the computer-based systems of the bioreactor of the present disclosure may apply supportive data to show killing activity by metabolic changes.

In some embodiments, cancer drug resistance/activity testing using metabolic changes readout for testing an anti-cancer drug may be used to determine whether the targeted drug stops or slows the tissue culture by measuring a change in the metabolism-correlating drug resistance and/or activity testing.

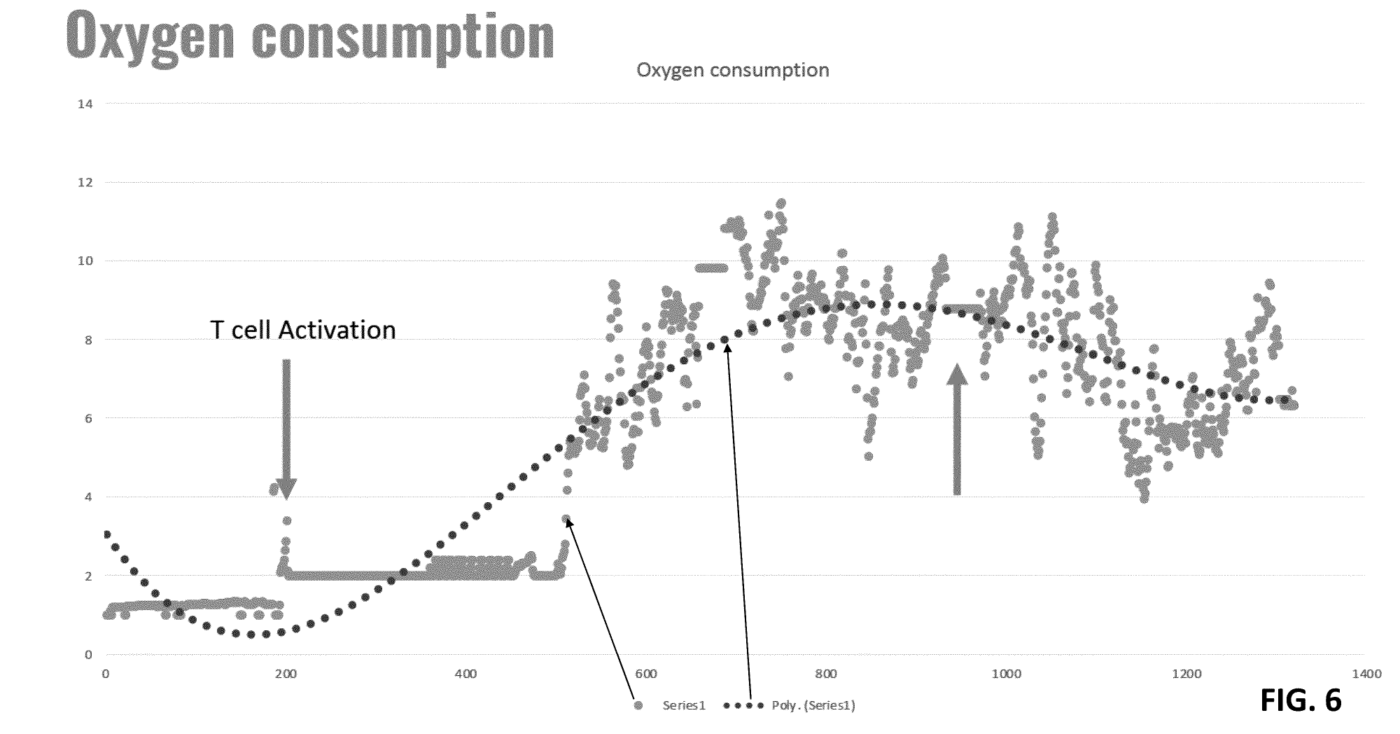

FIG. 6 is a graph showing an oxygen consumption level (DO) versus run time in accordance with one or more embodiments of the present invention. The graph shows a correlation from time of activation (left arrow) over the run time with changes in oxygen consumption during the run for a plurality of samples. As the oxygen increases, at the right arrow, the process slows down due to metabolic changes, and not due to cell death. The viability is 96%.

FIG. 7 is a graph showing a lactate accumulation rate versus run time in accordance with one or more embodiments of the present invention. The graph shows a correlation from time of activation (at the arrow) over the run time with changes in the lactate accumulation rate during the run.

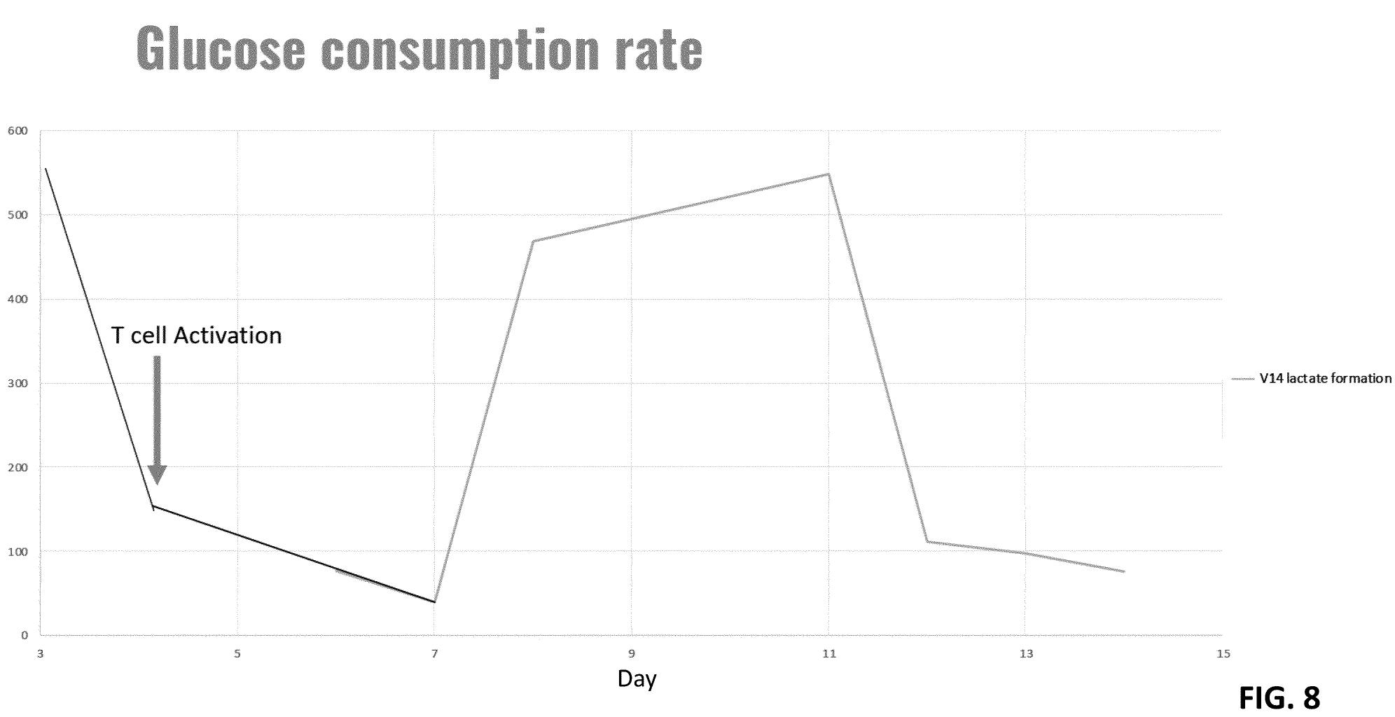

FIG. 8 is a graph showing a glucose consumption rate versus run time in accordance with one or more embodiments of the present invention. The graph shows a correlation from time of activation (arrow) over the run time with changes in glucose consumption rate during the run.

Generally, it would be known by one skilled in the art that a machine learning model may be trained using training datasets that would have specific input data features and specific output data features to fit the machine learning model for outputting the specific output data features within a desired predictive accuracy in response to inputting the specific input data features. Stated differently, the trained machine learning model maps the specific input data features to the specific output data features. Consequently, without the above detailed specificity of (1) a particular machine learning model and (2) a particular training dataset having particular input and output, any training process would fail to achieve convergence to provide predictive accuracy.

In some embodiments, the exemplary parametric correlations over time shown in FIGS. 6-8 over thousands of cell culture runs for thousands of subjects (e.g., patients) may be utilized in constructing datasets capturing multi-parametric time-dependent correlations between the plurality of sensor parameters and the plurality of cell culture growth parameters. These specific input features in the datasets may include a desired cell growth configuration (e.g., an initialization and definitions file) and the sensor data from each of the plurality of sensors (e.g., different sensor types such as pH, DO, temperature, etc.) at different sampling times as well as imaging data. The desired cell growth configuration may include at least one cell culture growth parameter from a plurality of cell culture growth parameters, and at least one desired range for each of the plurality of sensor parameters measured by the plurality of sensors at predefined time intervals for each of the at least one cell culture growth parameter during the growth of cell culture. In other embodiments, the at least one desired range may include single parametric setpoints.

The specific output features in the datasets may include a plurality of output data features related to cell culture parameter predictions and/or output features that may be include a plurality of cell culture control actions such as but not limited to transmitting at least one control circuitry command (e.g., a digital command to the control circuitry 230) to one or more control devices to control: a display, a gas level and/or a liquid flow in the bioreactor chamber, and/or to remove waste products from the liquid medium.

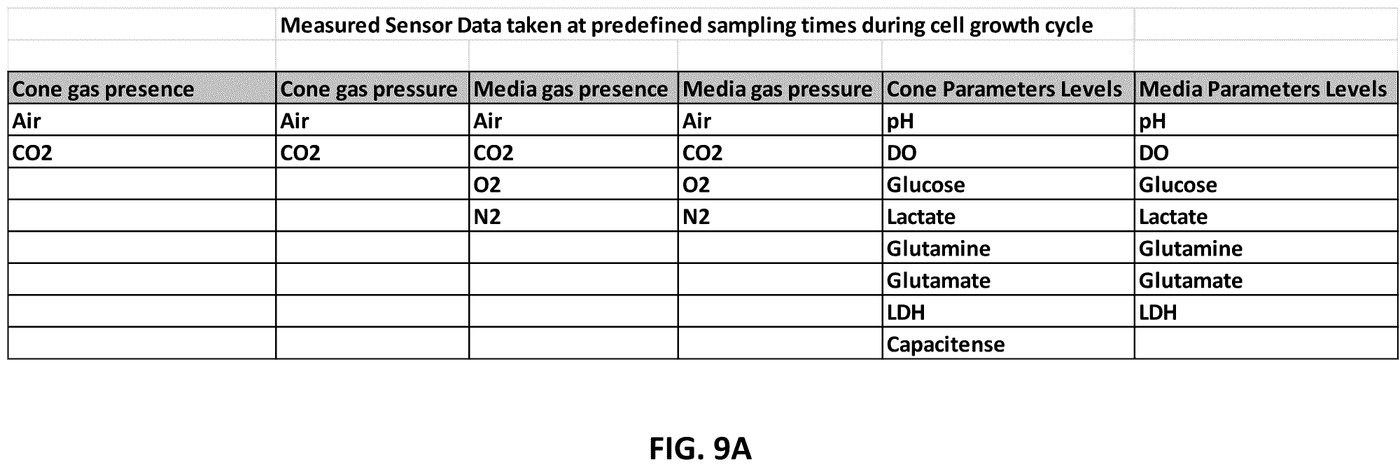

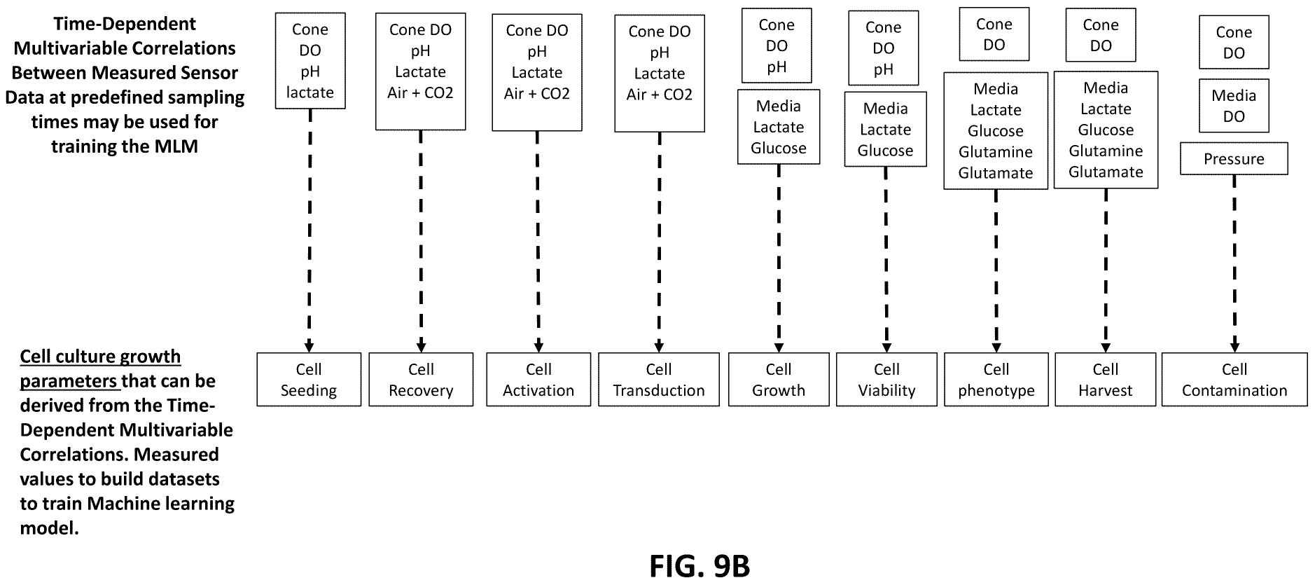

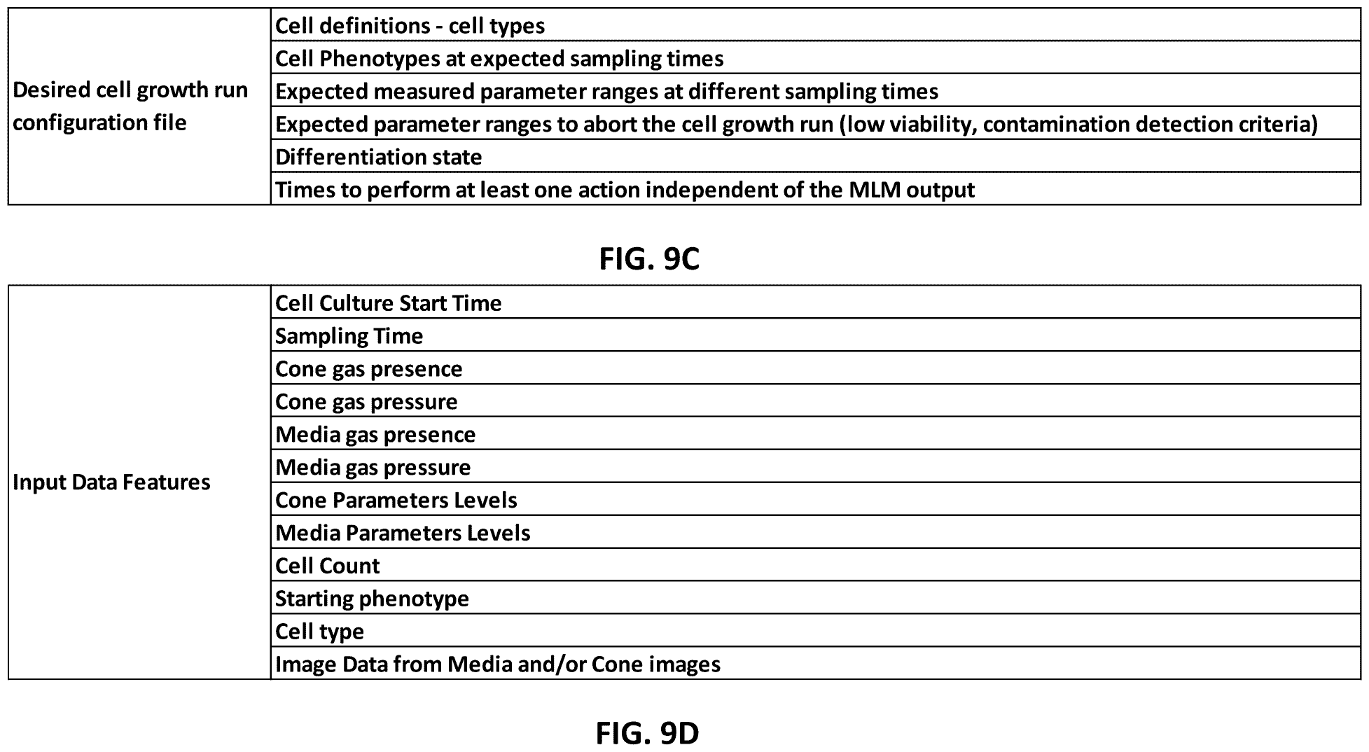

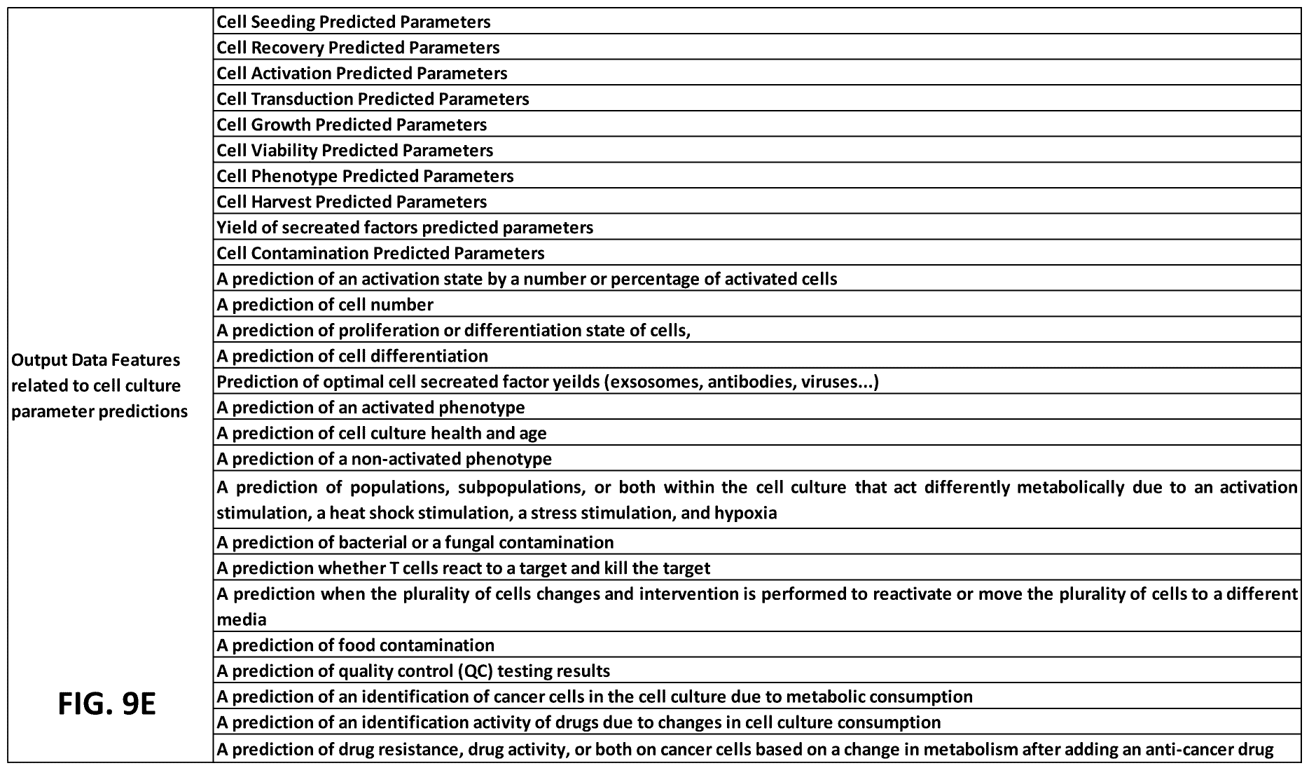

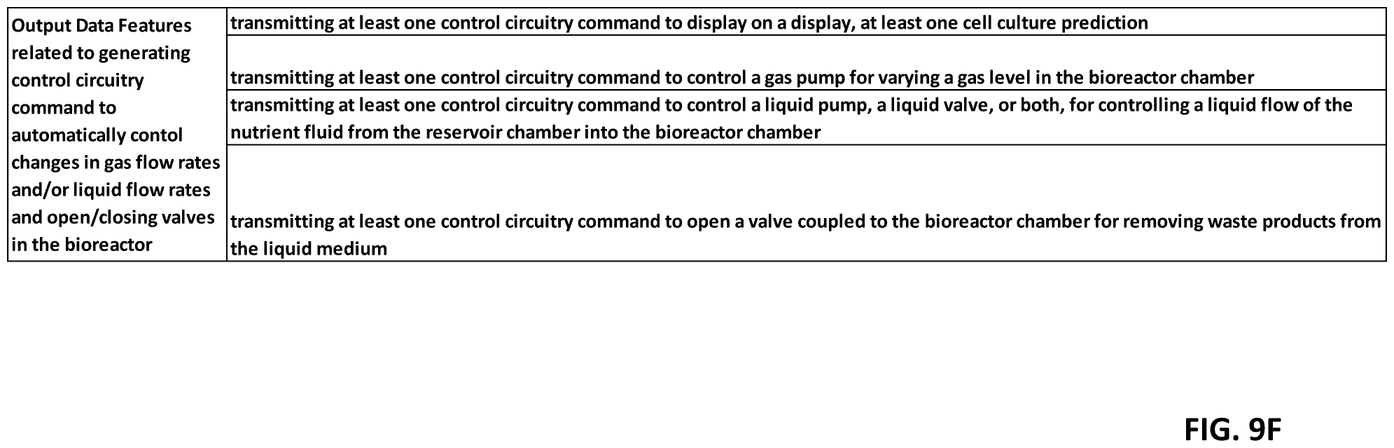

FIGS. 9A-9E are tables shown different parameters features whose values may be used to construct datasets for training at least one machine learning model. These tables may be related to, but not limited to the bioreactor chamber embodiment discussed hereinabove of a cone shaped bioreactor chamber partitioned by a perforated barrier into two sub chambers-a cone sub-chamber for cell growth and a media sub-chamber. Each sub-chamber may have its own sensors and computer-controlled devices for controlling a gas flow and/or a fluid flow in the sub-chambers.

FIG. 9A is a table showing the measured sensor data levels in the cone and the media sub-chambers at predefined sampling times during the cell growth cycle in accordance with one or more embodiments of the present disclosure.

FIG. 9B is a diagram showing correlations between the measured sensor data and cell culture growth parameters in accordance with one or more embodiments of the present disclosure. Time dependent multivariable correlations between the measured sensor data in the cone and media at the predefined sampling times may be used determine cell culture growth parameters that can be derived from these time dependent correlations. For example, from FIG. 9B, correlations between the measured cone DO, pH and lactate levels measured by the sensors at different sampling times (from the start time of the cell growth process) may be used to monitor cell seeding. Similarly, correlations between the measured cone DO level, the media lactate level, the media glucose level, the media glutamine level, and the media glutamate level at different sampling times may be used to determine cell phenotype and cell harvest. These correlations may be used to build datasets to train a machine learning model for a plurality of sampling time from cell run start time to the cell run end time over a plurality of cell samples taken from a respective plurality of subjects (e.g., patients, for example).

In some embodiments, a correlation between two or more parameters (e.g., oxygen/lactate/glucose consumption) may represent the cell stage where for example, post activation glucose slows down, lactate goes up and oxygen goes up as well, where the parametric correlations may be based on measuring differences in the slope. For example, at day 11, the metabolism changes and cells are no longer activated-exemplified by the change in the profile of all three parameters.

In some embodiments, datasets capturing correlations among various parameters over time that detected and monitored in real time by the sensors 104a, 104b, and 104c, for example, may be used to train the cell culture control machine learning model 205 to predict the cellular conditions and/or outcomes at different stages of the growth process. As a result, these correlations between cell conditions and specific measured parameters may provide future estimations related to cell quantity, cell health, and other factors. Moreover, if certain parameters at a particular time in the growth process may have been determined to result in poor cell health at a particular future time and the inefficacy to obtain good cells for harvesting, the MLM may output, for example, a warning to abort the particular cell growth process and/or a set of commands via the control circuitry to automatically control the bioreactor fluid control devices and/or temperature controller to change the system parameters to place the cell growth on a different path for obtaining good cell health such that the cells may be harvested in the future.

In some embodiments, the at least one cell culture control MLM 205 may be trained with parametric correlations between at least 2 and 3 parameters. In some embodiments, the at least one cell culture control MLM 205 may be trained with parametric correlations between at least 3 and 4 parameters. In some embodiments, the at least one cell culture control MLM 205 may be trained with parametric correlations between at least 4 and 6 parameters. In some embodiments, the at least one cell culture control MLM 205 may be trained with parametric correlations between at least 6 and 10 parameters. In some embodiments, the at least one cell culture control MLM 205 may be trained with parametric correlations between at least 10 parameters. In some embodiments, the at least one cell culture control MLM 205 may be trained with parametric correlations between at least 50 parameters.

FIG. 9C is a table showing desired cell growth run configuration file parameters in accordance with one or more embodiments of the present disclosure. These parameters may be entered into the system when starting a cell growth run (e.g., an initialization configuration data file). These parameters may include for example, performing one action independent of the machine learning model output. Furthermore, these parameters may be used in building datasets for training the at least one cell culture control MLM 205.

FIG. 9D is a table showing input data features for the cone bio-reactor chamber in accordance with one or more embodiments of the present disclosure. The input data features may include the cell culture start time, the sampling time after the cell culture start time, cone and/or media gas parameters, cone and/or media parameter levels (DO, pH, CO2, O2, N2, glucose, Lactate, Glutamine, Glutamate as in FIG. 9A), cell count and/or image data from the media and/or cone images.

In some embodiments, the computer-based systems of the bioreactor of the present disclosure may be used to build a plurality of training datasets based on a correlation between the different slopes of different parameters such as but not limited to O2, Glu, Lac, pH as well as the cell parameters and system parameters at different times t where t=0 may be the start of the growth process so as to predict the state the cells and their growth process at future times. The at least one cell culture control machine learning model 205 may be trained to output a cell culture parameter prediction of one or more of the following based on monitoring different parameters:
1. Prediction of activation state and number or percentage of activated cells
2. Prediction of cell number
3. Prediction of proliferation or differentiation state of cells.
4. Prediction of cell differentiation
5. Prediction of phenotype (activated/non activated)
6. Prediction of population and subpopulations within the culture that act differently metabolically due to stimulation such as activation, heat shock, stress, hypoxia . . .
7. Prediction of contamination (bacteria or fungus)
8. Prediction or even testing of functionality (do the T cells react to the target and kill it)
9. Prediction when the cells change and intervention may be needed such as to reactivate or move to a different media.
10. Predictions of food contamination. This can be used in other industry such as food to see contamination.
11. Predictions of quality control (QC) testing which can be done inside the device as they grow including phenotype, activity, and sterility. The QC concept is using indirect measurements and correlations to test activity and phenotype.
12. Predictions regarding the identification of cancer cells in a culture due to metabolic consumption.
13. Predictions regarding the identification activity of drugs such as antibiotics or anticancer by the changes in consumption.
14. Prediction of drug resistance and drug activity on cancer cells—for example, a change in metabolism due to addition of an anti-cancer drug.

FIG. 9E a table showing output data features for the cone bio-reactor chamber related culture to cell parameter predictions in accordance with one or more embodiments of the present disclosure. The input data features of FIG. 9D coupled with the output data features of FIG. 9E may be used to generate datasets at different sampling times and over a plurality of cell samples taken from a respective plurality of subjects to generate datasets with millions of data features for training the at least one cell culture control MLM 205. The predicted values of the output data features shown in FIG. 9E may be based on the at least one cell culture control MLM 205 being trained with measured data from FIG. 9B.

In some embodiments, the at least one cell culture control MLM 205 may output predicted values for a particular output data feature as in FIG. 9E with a predictive accuracy of 0.1-1% relative to the measured values in the dataset depending on the type of predicted parameter chosen. In other embodiments, the at least one cell culture control MLM 205 may output predicted values for a particular output data feature as in FIG. 9E with a predictive accuracy of 1-10% relative to the measured values in the dataset depending on the type of predicted parameter chosen. In yet other embodiments, the at least one cell culture control MLM 205 may output predicted values for a particular output data feature as in FIG. 9E with a predictive accuracy of 10-30% relative to the measured values in the dataset depending on the type of predicted parameter chosen.

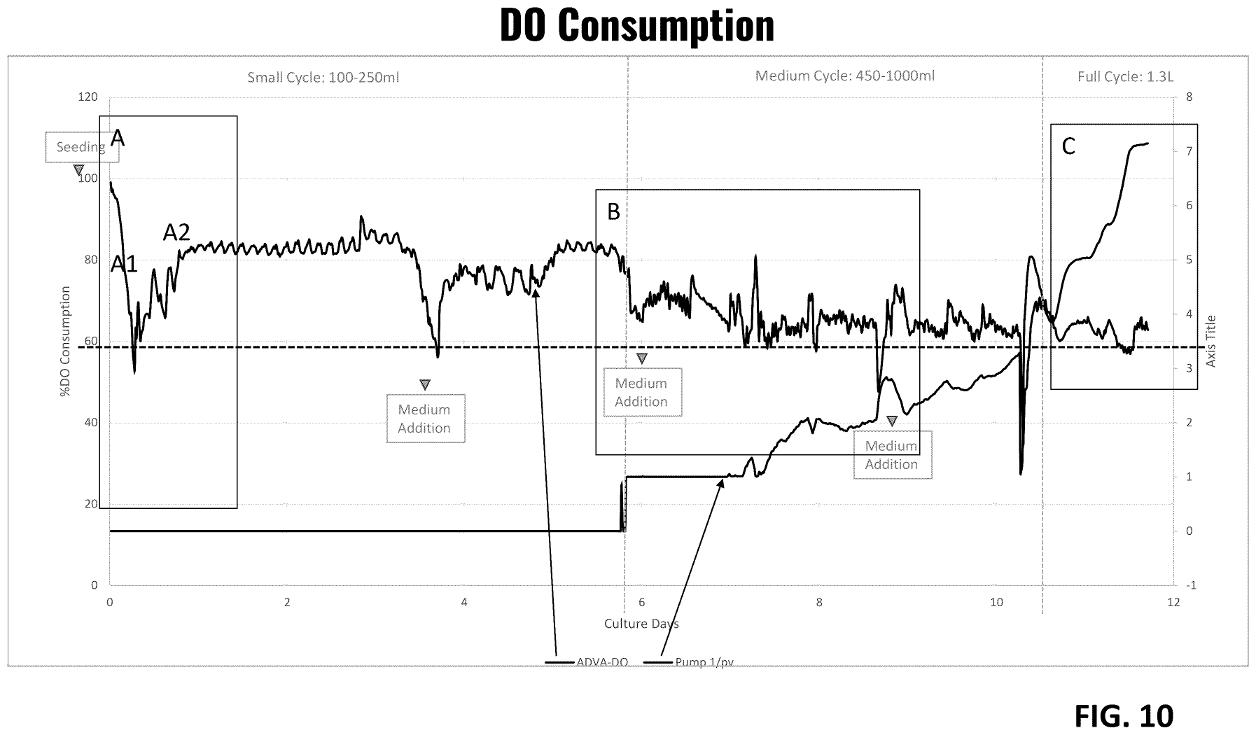

In some embodiments, the different predictions or prediction outcomes shown in FIG. 9E may be in the form of a flag-Yes, contamination, or NO contamination based on the input sensor levels readings over multiple sampling times and the correlations between them. In other embodiments, prediction may include predicted values and/or predicted ranges of values based on the measured values for each of the output metrics at future times based on the correlations such as shown previously in FIGS. 2-8 and in later figures (FIG. 10 to FIGS. 16A and 16B).

FIG. 9F is a table of output data features from the at least one cell culture control MLM 205 that transmit output commands to the control circuitry 230 in accordance with one or more embodiments of the present disclosure. At least one control circuitry command may be used by the control circuitry 230 to automatically control changes in gas flow rates and/or liquid flow rates and/or waste removal in the bioreactor chamber.

In some embodiments, the at least one control circuitry command may cause a display to display, at least one cell culture prediction (e.g., any of the predictions in FIG. 9E).

In some embodiments, the at least one control circuitry command may control a gas control device for varying a gas level in the bioreactor chamber.

In some embodiments, the at least one control circuitry command may control a device for controlling a liquid flow of the nutrient fluid from the reservoir chamber into the bioreactor chamber.

In some embodiments, the at least one control circuitry command may control a device coupled to the bioreactor chamber for removing waste products from the liquid medium, for example.

In some embodiments, the at least one cell culture control MLM 205 may be further trained to output automated actions in the bioreactor system 100A and/or 100B such as in the following scenarios.

In some embodiments, stem cells may proliferate in a bioreactor on a surface or in suspension. Once the stem cells reach confluence or a desired density, their growth may slow down or stop expending, which in turn may change the glucose, oxygen and/or lactate accumulation rate, changing the ratios between them. At this point, the at least one cell culture control MLM 205 may cause the system to add cytokines and/or differentiation factors to the cell culture so as to trigger the differentiation to the required phenotype.

In some embodiments, cells such as Mesenchymal stem cells may secrete extra cellular vesicles called Exosomes. The seeded cells in the bioreactor may grow until reaching the particular density or activity as defined by the ratios of the metabolic parameters. Once the particular density may be reached, the media may be automatically changed by at least one control command outputted from the at least one cell culture control MLM 205 to control devices to change the media from a rich growth supporting media to a collection media which may be serum free so as to insure high yields of harvested exosomes. The same scenario may occur for vaccines, viruses, proteins, and/or antibody secretion, where the at least one control command outputted from the at least one cell culture control MLM 205 may be used to optimizing yields.

In some embodiments, mesenchymal stem cells may grow until confluence. and then the Oxygen level in the system may be automatically changed by at least one control command outputted from the at least one cell culture control MLM 205 to control devices to prime cells to a hypoxia state, which has been shown to lead to a more potent activity of angiogenesis.

In some embodiments, cell contamination may be identified by measurements taken over consecutive time samples showing correlations of sudden changes in pH, sudden drop in DO, glucose, lactate, pressure, and/or change of liquid medium color parameters (e.g., the liquid medium may turn cloudy), for example. Data from thousand of cell samples that were prematurely terminated due to contamination may be used to train the at least one cell culture control MLM 205 to identify cell contamination. Thus, when real time measured sensor and/or image data at predefined sampling times for a cell culture may be input into the at least one cell culture control MLM 205 trained with these correlations, the at least one cell culture control MLM 205 may output a prediction that the cell culture is contaminated and generate commands to display a warning to a bioreactor system operator or sound an alarm.

In some embodiments, the at least one cell culture control MLM 205 may be further trained to generate control circuitry commands to perform automatic system corrections by initiating changes in gas and/or liquid levels in the bio-reactor chamber shown in FIG. 9E. For example, if over consecutive time samples, a drop in DO or for example a change in the lactate formation rate may be observed indicating the cells may not be reacting as expected, the at least one cell culture control MLM 205 may detect this and output a command to the control devices to automatically introduce Interleukin-2 (e.g., IL-2) into the cell sample so as to increase for example, T-cell proliferation. The at least one cell culture control MLM 205 may automatically monitor the dosing schedule of IL-2 over time so as to prevent T-cell exhaustion and optimize cell stimulation.

The cell culture control machine learning model 205 may include types of machine learning models that may include, but are not limited to, decision trees, boosting, support-vector machines, neural networks, nearest neighbor algorithms, Naive Bayes, bagging, random forests, and the like. In some embodiments and, optionally, in combination of any embodiment described above or below, an exemplary neutral network technique may be one of, without limitation, feedforward neural network, radial basis function network, recurrent neural network, convolutional network (e.g., U-net) or other suitable network.

In some embodiments and, optionally, in combination of any embodiment described above or below, an exemplary implementation of Neural Network may be executed as follows:
i) Define Neural Network architecture/model,
ii) Transfer the input data to the exemplary neural network model,
iii) Train the exemplary model incrementally,
iv) determine the accuracy for a specific number of timesteps,
v) apply the exemplary trained model to process the newly-received input data,
vi) optionally and in parallel, continue to train the exemplary trained model with a predetermined periodicity.

In some embodiments and, optionally, in combination of any embodiment described above or below, the exemplary trained neural network model (e.g., MLM 205) may specify a neural network by at least a neural network topology, a series of activation functions, and connection weights. For example, the topology of a neural network may include a configuration of nodes of the neural network and connections between such nodes. In some embodiments and, optionally, in combination of any embodiment described above or below, the exemplary trained neural network model may also be specified to include other parameters, including but not limited to, bias values/functions and/or aggregation functions. For example, an activation function of a node may be a step function, sine function, continuous or piecewise linear function, sigmoid function, hyperbolic tangent function, or other type of mathematical function that represents a threshold at which the node is activated. In some embodiments and, optionally, in combination of any embodiment described above or below, the exemplary aggregation function may be a mathematical function that combines (e.g., sum, product, etc.) input signals to the node. In some embodiments and, optionally, in combination of any embodiment described above or below, an output of the exemplary aggregation function may be used as input to the exemplary activation function. In some embodiments and, optionally, in combination of any embodiment described above or below, the bias may be a constant value or function that may be used by the aggregation function and/or the activation function to make the node more or less likely to be activated.

In some embodiments, the bioreactor system 100 may be configured to automatically construct datasets with collected data from cell sample runs and measured data. These datasets may be used by the controller 106 to automatically retrain the at least one cell culture control machine learning model 205. Optionally or alternatively, datasets from a plurality of bioreactor systems 100 with cell cultures from a respective plurality of subjects (e.g., patients) may be transmitted via communication circuitry 220 to a backend cloud server (not shown) to retrain the at least one cell culture control machine learning model 205 using the datasets from the different bioreactor systems.

In some embodiments, the at least one cell culture control MLM 205 may include two machine learning models where a first machine learning model may be trained to map input data including sensor data from each of the plurality of sensors to output data that includes cell culture predicted parameters. A second machine learning model may map the cell culture predicted parameters to a plurality of control circuitry commands to automatically control gas flow and/or liquid flow in the bioreactor chamber.

In some embodiments, metabolic analyses such as correlations in changes in the ratios between factors of DO, Glucose, Lactate, Glutamine, and/or glutamate levels over time during cell culture growth may enable the identification of metabolic alterations, that may be crucial for the T-cells (Effector and Regulatory cells) differentiation and functionality. For example, effector T-cells may increase a glycolysis level upon activation. Measuring changes in levels of glucose consumption and lactate production rates may be key indicators for expansion progress. Regulatory T-cells may have different metabolic preferences such as for example, Fatty Acid Oxidation and mitochondrial metabolism that may be more dominant in Regulatory T-cells than in effector T-cells. Glutamine metabolism may be involved in regulating the expansion and function of Regulatory t-cells. Glutamine uptake may be massively enhanced upon TCR stimulation in T lymphocytes and may be crucial for their proliferation and cytokine production.

FIG. 10 are graphs showing a phenotypic prediction using metabolic sensing in accordance with one or more embodiments of the present disclosure. In day 1 (region A), a mixture of PBMC (peripheral blood mononuclear cells) is non-activated showing a O2 consumption with a steep slope at marker A1. At marker A2, a large subset of cells such as non-T cells have died, as indicated by a lower O2 consumption. Once the T cells have been activated at Day 6 (region B), higher O2 consumption due to proliferation results and the O2 pump rate is increased to keep the DO level substantially flat as the T-grow with higher O2 consumption (Region C). (Top trace is the O2 feed, Bottom trace is O2 level).

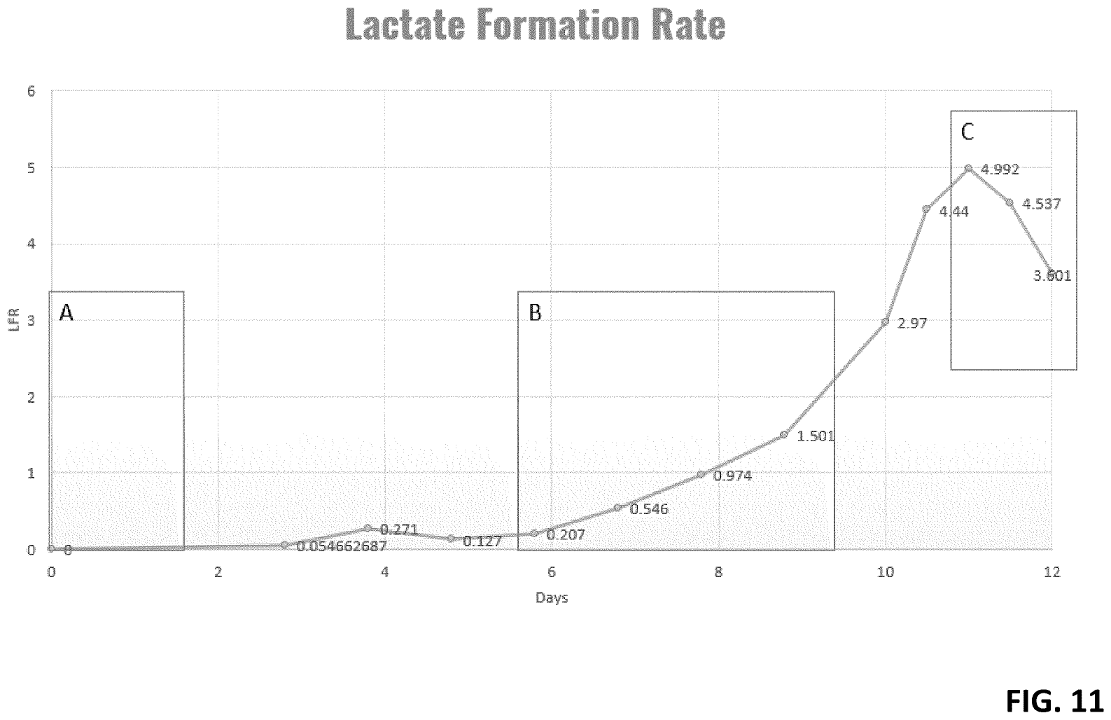

FIG. 11 is a graph showing lactate formation in accordance with one or more embodiments of the present disclosure. Prior to the activation of T cells at Day 6 (region A), there is minimal Lactate formation. At Day 6 (Region B), the Activated T cells start to generate lactate and the lactate formation rate increases. At Day 11 (Region C), the T cells change their phenotype to non-activated and the lactate formation rate drops.

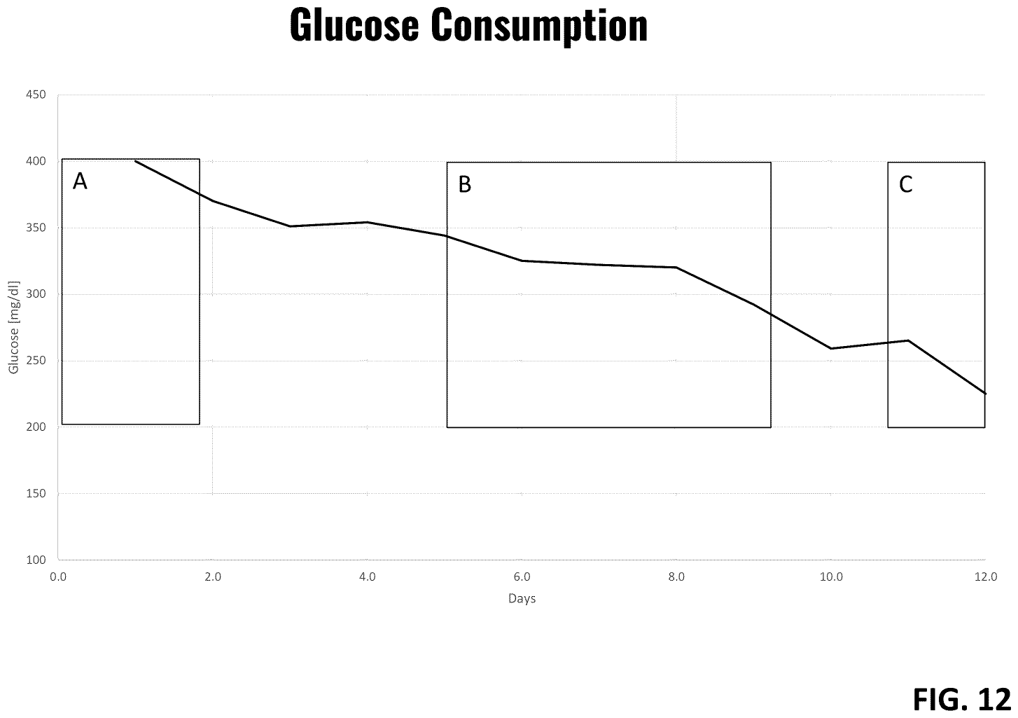

FIG. 12 is a graph showing glucose consumption in accordance with one or more embodiments of the present disclosure. In region A, there is no growth so minimum glucose consumption is observed (higher DO level). In region B, the T-cells are activated and started to grow resulting in higher glucose consumption as the DO levels drop. In region C, there is a change in phenotype in the T-cells from activated to non-activated as observed as a higher glucose consumption as the DO levels drop further.

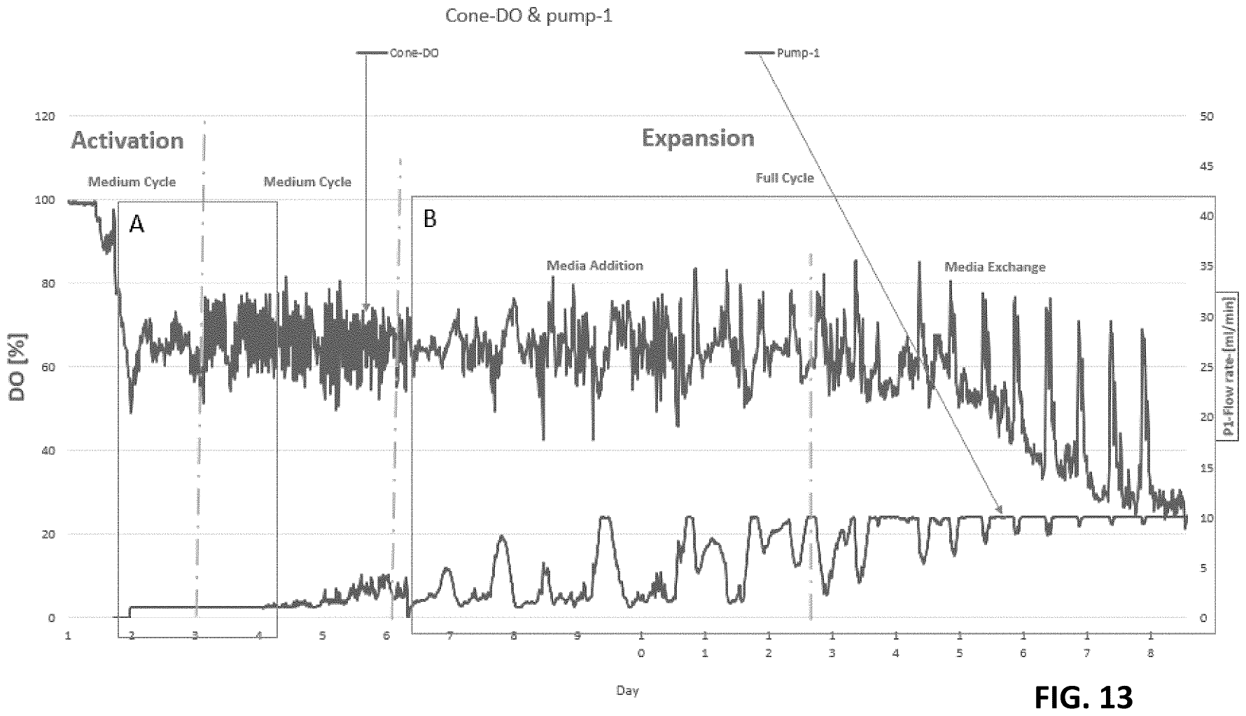

FIG. 13 is a graph showing dissolved oxygen in the cone and the O2 pump rate in accordance with one or more embodiments of the present disclosure. In a pre-activation region A, there is a slight increase in oxygen consumption with a nearly flat and low O2 pump rate. In a post-activation region B, media is added to the cone with DO levels spike around 60% and at around Day 13, when the media is exchanged, there an increase oxygen consumption with a higher O2 pump rate.

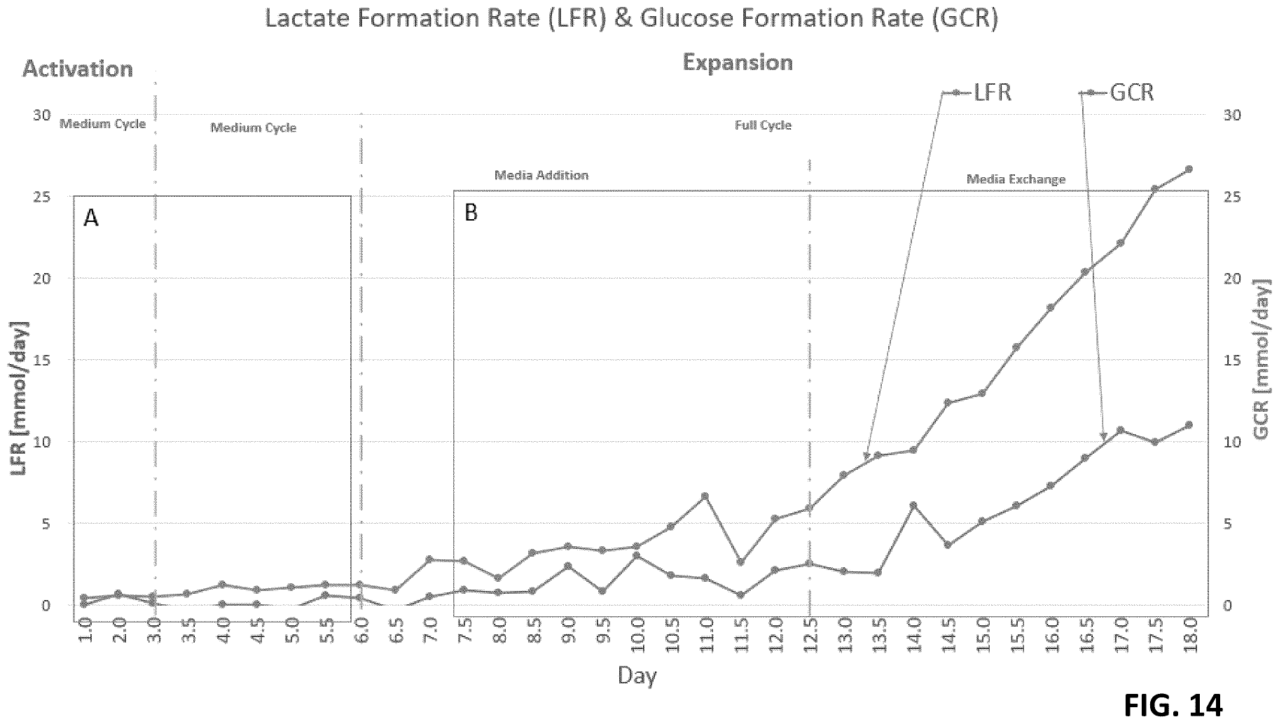

FIG. 14 is a graph showing lactate formation rate (LFR) and glucose formation rate (GCR) in accordance with one or more embodiments of the present disclosure. In pre-activation region A, there is a slight increase in LFR and GCR. In the activation region B, the lactate levels increase and the glucose level slows down per cell particularly as the media is exchanged.

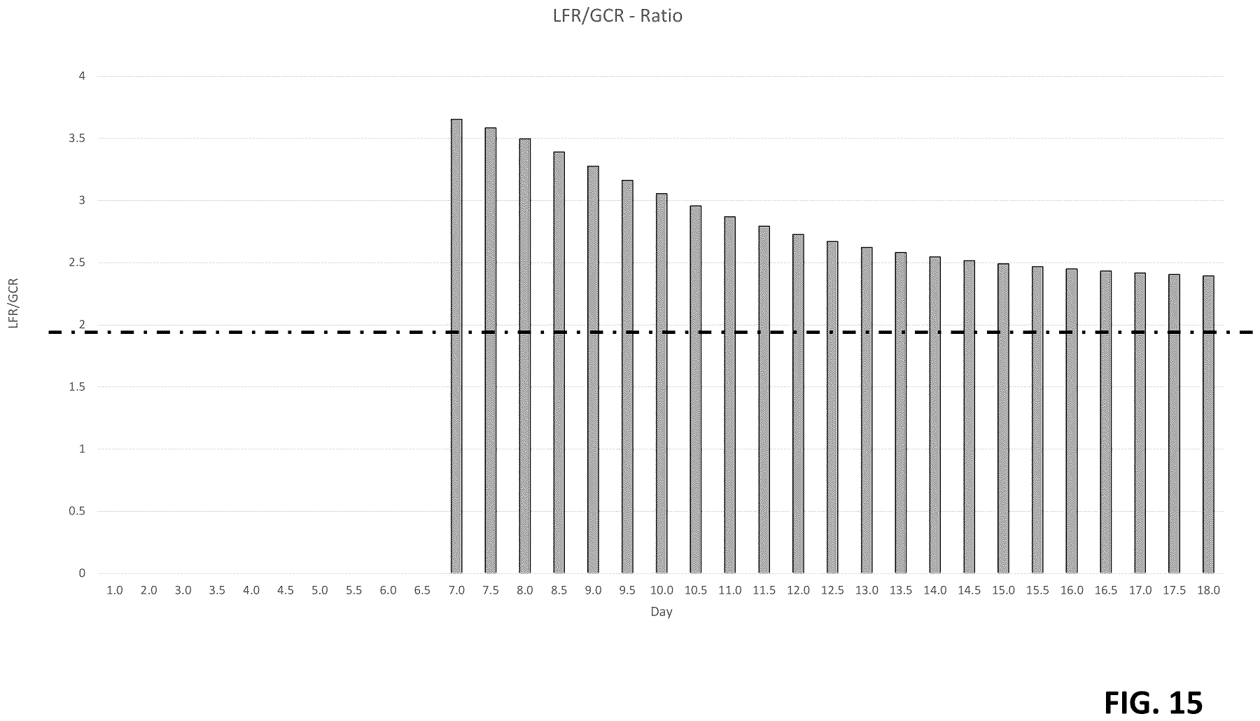

FIG. 15 is a graph showing an LFR/GCR ratio in accordance with one or more embodiments of the present disclosure. As the culture expends, the population may change and the ratio between Glucose and Lactate changes with more activated cells. Thus, FIG. 13-15 shows that each of the parameter reacts different during the run. The ratios change showing the change of state of the plurality of cells. FIG. 15 shows that the LFR/GCR ratio almost reaching a theoretical ratio value of 2 indicating that the nearly 100% of the cells may be in an expansion state.

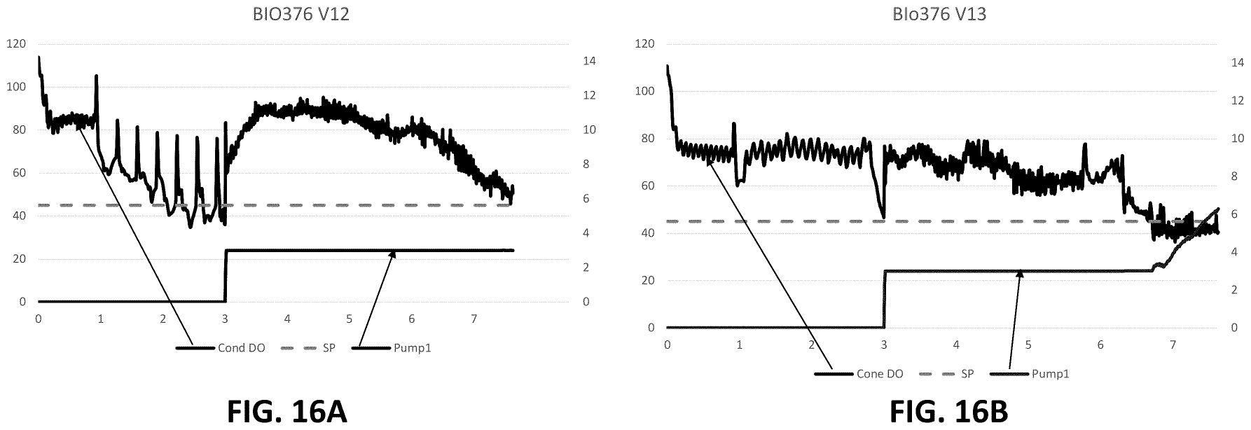

FIGS. 16A and 16B show two embodiments of transduction efficiency in accordance with one or more embodiments of the present disclosure. Following cell transduction, perfusion begins after 24 hours where the dissolved oxygen percentage may increase to higher values when transduction efficiency is low but stays at a lower percentage when transduction efficiency is high. When the DO falls below the DO set point (SP), the O2 pump may be triggered causing a rise in DO (Day 3). Additionally, the pump rate and DO level may exhibit a higher increase rate in cases of low transduction efficiency compared to those with high transduction efficiency. In FIG. 16B, there is a small population of cells activated as evidenced by the DO drop.

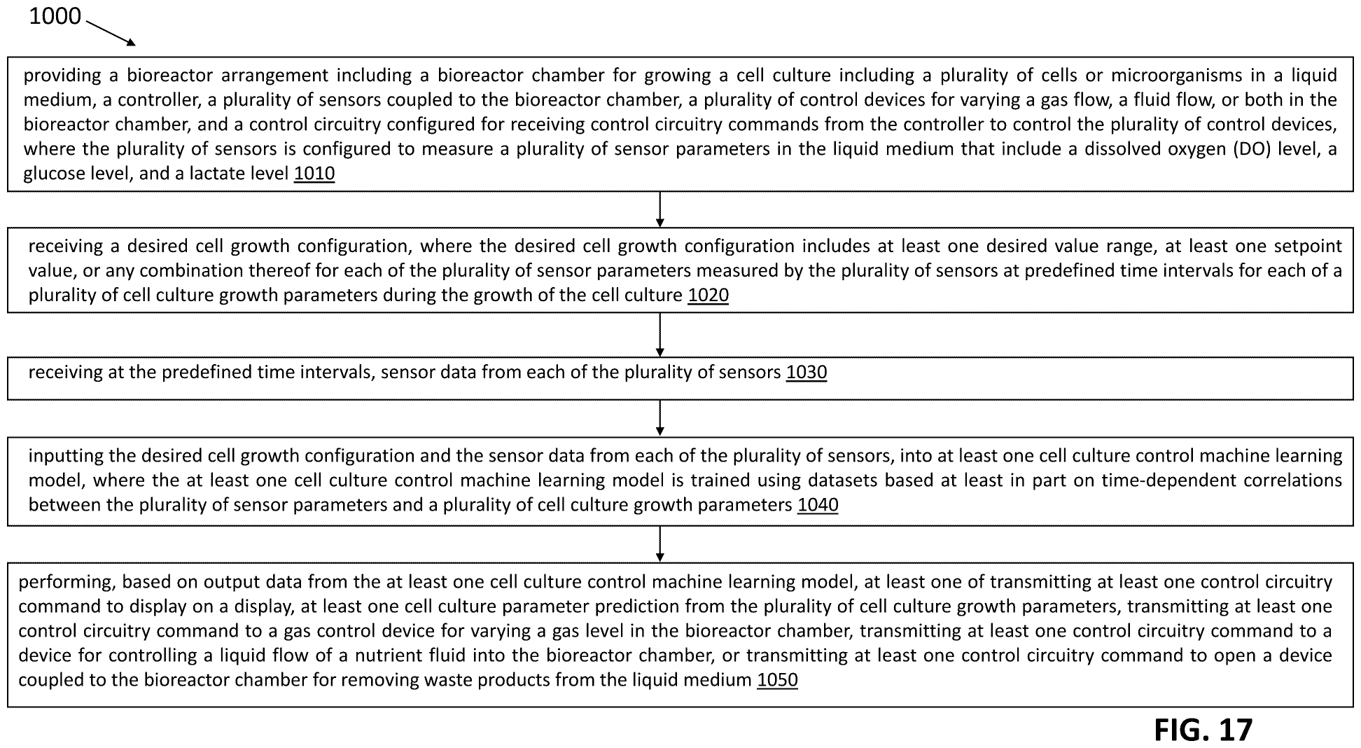

FIG. 17 is flowchart of a method 1000 for controlling and monitoring metabolic rate and environmental factors of at least one bioreactor in accordance with one or more embodiments of the present disclosure.

The method 1000 may include providing 1010 a bioreactor arrangement including a bioreactor chamber for growing a cell culture including a plurality of cells or microorganisms in a liquid medium, a controller, a plurality of sensors coupled to the bioreactor chamber, a plurality of control devices for varying a gas flow, a fluid flow, or both in the bioreactor chamber, and a control circuitry configured for receiving control circuitry commands from the controller to control the plurality of control devices, where the plurality of sensors is configured to measure a plurality of sensor parameters in the liquid medium that include a dissolved oxygen (DO) level, a glucose level, and a lactate level.

The method 1000 may include receiving 1020, by the controller 106, a desired cell growth configuration, where the desired cell growth configuration includes at least one desired value range, at least one setpoint value, or any combination thereof for each of the plurality of sensor parameters measured by the plurality of sensors at predefined time intervals for each of a plurality of cell culture growth parameters during the growth of the cell culture.

The method 1000 may include receiving 1030, by the controller 106, at the predefined time intervals, sensor data from each of the plurality of sensors.

The method 1000 may include inputting 1040, by the controller 106, the desired cell growth configuration and the sensor data from each of the plurality of sensors, into at least one cell culture control machine learning model, where the at least one cell culture control machine learning model is trained using datasets based at least in part on time-dependent correlations between the plurality of sensor parameters and a plurality of cell culture growth parameters.

The method 1000 may include performing 1050, by the controller 106, based on output data from the at least one cell culture control machine learning model, at least one of transmitting at least one control circuitry command to display on a display, at least one cell culture parameter prediction from the plurality of cell culture growth parameters, transmitting at least one control circuitry command to a control a gas control device for varying a gas level in the bioreactor chamber, transmitting at least one control circuitry command to a device for controlling a liquid flow of a nutrient fluid into the bioreactor chamber, or transmitting at least one control circuitry command to open a device coupled to the bioreactor chamber for removing waste products from the liquid medium.

In some embodiments, exemplary inventive, specially programmed computing systems/platforms with associated devices are configured to operate in the distributed network environment, communicating with one another over one or more suitable data communication networks (e.g., the Internet, satellite, etc.) and utilizing one or more suitable data communication protocols/modes such as, without limitation, IPX/SPX, X.25, AX.25, AppleTalk™, TCP/IP (e.g., HTTP), near-field wireless communication (NFC), RFID, Narrow Band Internet of Things (NBIOT), 3G, 4G, 5G, GSM, GPRS, WiFi, WiMax, CDMA, satellite, ZigBee, and other suitable communication modes. In some embodiments, the NFC can represent a short-range wireless communications technology in which NFC-enabled devices are “swiped,” “bumped,” “tap” or otherwise moved in close proximity to communicate. In some embodiments, the NFC could include a set of short-range wireless technologies, typically requiring a distance of 10 cm or less. In some embodiments, the NFC may operate at 13.56 MHz on ISO/IEC 18000-3 air interface and at rates ranging from 106 kbit/s to 424 kbit/s. In some embodiments, the NFC can involve an initiator and a target; the initiator actively generates an RF field that can power a passive target. In some embodiments, this can enable NFC targets to take very simple form factors such as tags, stickers, key fobs, or cards that do not require batteries. In some embodiments, the NFC's peer-to-peer communication can be conducted when a plurality of NFC-enable devices (e.g., smartphones) within close proximity of each other.

The material disclosed herein may be implemented in software or firmware or a combination of them or as instructions stored on a machine-readable medium, which may be read and executed by one or more processors. A machine-readable medium may include any medium and/or mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others.

As used herein, the terms “computer engine” and “engine” identify at least one software component and/or a combination of at least one software component and at least one hardware component which are designed/programmed/configured to manage/control other software and/or hardware components (such as the libraries, software development kits (SDKs), objects, etc.).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Computer-related systems, computer systems, and systems, as used herein, include any combination of hardware and software. Examples of software may include software components, operating system software, middleware, firmware, software modules, routines, subroutines, functions, methods, procedures, software interfaces, application program interfaces (API), instruction sets, computer code, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores” may be stored on a tangible, machine readable medium and supplied to various customers or manufacturing facilities to load into the fabrication machines that make the logic or processor. Of note, various embodiments described herein may, of course, be implemented using any appropriate hardware and/or computing software languages (e.g., C++, Objective-C, Swift, Java, JavaScript, Python, Perl, QT, etc.).

In some embodiments, one or more of exemplary inventive computer-based systems/platforms, exemplary inventive computer-based devices, and/or exemplary inventive computer-based components of the present disclosure may include or be incorporated, partially or entirely into at least one personal computer (PC), laptop computer, ultra-laptop computer, tablet, touch pad, portable computer, handheld computer, palmtop computer, personal digital assistant (PDA), cellular telephone, combination cellular telephone/PDA, television, smart device (e.g., smart phone, smart tablet or smart television), mobile internet device (MID), messaging device, data communication device, and so forth.

As used herein, the term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

In some embodiments, as detailed herein, one or more of exemplary inventive computer-based systems/platforms, exemplary inventive computer-based devices, and/or exemplary inventive computer-based components of the present disclosure may obtain, manipulate, transfer, store, transform, generate, and/or output any digital object and/or data unit (e.g., from inside and/or outside of a particular application) that can be in any suitable form such as, without limitation, a file, a contact, a task, an email, a social media post, a map, an entire application (e.g., a calculator), etc. In some embodiments, as detailed herein, one or more of exemplary inventive computer-based systems/platforms, exemplary inventive computer-based devices, and/or exemplary inventive computer-based components of the present disclosure may be implemented across one or more of various computer platforms such as, but not limited to: (1) FreeBSD, NetBSD, OpenBSD; (2) Linux; (3) Microsoft Windows; (4) OS X (MacOS); (5) MacOS 11; (6) Solaris; (7) Android; (8) iOS; (9) Embedded Linux; (10) Tizen; (11) WebOS; (12) IBM i; (13) IBM AIX; (14) Binary Runtime Environment for Wireless (BREW); (15) Cocoa (API); (16) Cocoa Touch; (17) Java Platforms; (18) JavaFX; (19) JavaFX Mobile; (20) Microsoft DirectX; (21) .NET Framework; (22) Silverlight; (23) Open Web Platform; (24) Oracle Database; (25) Qt; (26) Eclipse Rich Client Platform; (27) SAP NetWeaver; (28) Smartface; and/or (29) Windows Runtime.

In some embodiments, exemplary inventive computer-based systems/platforms, exemplary inventive computer-based devices, and/or exemplary inventive computer-based components of the present disclosure may be configured to utilize hardwired circuitry that may be used in place of or in combination with software instructions to implement features consistent with principles of the disclosure. Thus, implementations consistent with principles of the disclosure are not limited to any specific combination of hardware circuitry and software. For example, various embodiments may be embodied in many different ways as a software component such as, without limitation, a stand-alone software package, a combination of software packages, or it may be a software package incorporated as a “tool” in a larger software product.

For example, exemplary software specifically programmed in accordance with one or more principles of the present disclosure may be downloadable from a network, for example, a website, as a stand-alone product or as an add-in package for installation in an existing software application. For example, exemplary software specifically programmed in accordance with one or more principles of the present disclosure may also be available as a client-server software application, or as a web-enabled software application. For example, exemplary software specifically programmed in accordance with one or more principles of the present disclosure may also be embodied as a software package installed on a hardware device.

In some embodiments, exemplary inventive computer-based systems/platforms, exemplary inventive computer-based devices, and/or exemplary inventive computer-based components of the present disclosure may be configured to handle numerous concurrent users that may be, but is not limited to, at least 100 (e.g., but not limited to, 100-999), at least 1,000 (e.g., but not limited to, 1,000-9,999), at least 10,000 (e.g., but not limited to, 10,000-99,999), at least 100,000 (e.g., but not limited to, 100,000-999,999), at least 1,000,000 (e.g., but not limited to, 1,000,000-9,999,999), at least 10,000,000 (e.g., but not limited to, 10,000,000-99,999,999), at least 100,000,000 (e.g., but not limited to, 100,000,000-999,999,999), at least 1,000,000,000 (e.g., but not limited to, 1,000,000,000-999,999,999,999), and so on.

In some embodiments, exemplary inventive computer-based systems/platforms, exemplary inventive computer-based devices, and/or exemplary inventive computer-based components of the present disclosure may be configured to output to distinct, specifically programmed graphical user interface implementations of the present disclosure (e.g., a desktop, a web app., etc.). In various implementations of the present disclosure, a final output may be displayed on a displaying screen which may be, without limitation, a screen of a computer, a screen of a mobile device, or the like. In various implementations, the display may be a holographic display. In various implementations, the display may be a transparent surface that may receive a visual projection. Such projections may convey various forms of information, images, and/or objects. For example, such projections may be a visual overlay for a mobile augmented reality (MAR) application.

As used herein, the term “mobile electronic device,” or the like, may refer to any portable electronic device that may or may not be enabled with location tracking functionality (e.g., MAC address, Internet Protocol (IP) address, or the like). For example, a mobile electronic device can include, but is not limited to, a mobile phone, Personal Digital Assistant (PDA), Blackberry™ Pager, Smartphone, or any other reasonable mobile electronic device.

As used herein, the terms “proximity detection,” “locating,” “location data,” “location information,” and “location tracking” refer to any form of location tracking technology or locating method that can be used to provide a location of, for example, a particular computing device/system/platform of the present disclosure and/or any associated computing devices, based at least in part on one or more of the following techniques/devices, without limitation: accelerometer(s), gyroscope(s), Global Positioning Systems (GPS); GPS accessed using Bluetooth™; GPS accessed using any reasonable form of wireless and/or non-wireless communication; WiFi™ server location data; Bluetooth™ based location data; triangulation such as, but not limited to, network based triangulation, WiFi™ server information based triangulation, Bluetooth™ server information based triangulation; Cell Identification based triangulation, Enhanced Cell Identification based triangulation, Uplink-Time difference of arrival (U-TDOA) based triangulation, Time of arrival (TOA) based triangulation, Angle of arrival (AOA) based triangulation; techniques and systems using a geographic coordinate system such as, but not limited to, longitudinal and latitudinal based, geodesic height based, Cartesian coordinates based; Radio Frequency Identification such as, but not limited to, Long range RFID, Short range RFID; using any form of RFID tag such as, but not limited to active RFID tags, passive RFID tags, battery assisted passive RFID tags; or any other reasonable way to determine location. For ease, at times the above variations are not listed or are only partially listed; this is in no way meant to be a limitation.

As used herein, the terms “cloud,” “Internet cloud,” “cloud computing,” “cloud architecture,” and similar terms correspond to at least one of the following: (1) a large number of computers connected through a real-time communication network (e.g., Internet); (2) providing the ability to run a program or application on many connected computers (e.g., physical machines, virtual machines (VMs)) at the same time; (3) network-based services, which appear to be provided by real server hardware, and are in fact served up by virtual hardware (e.g., virtual servers), simulated by software running on one or more real machines (e.g., allowing to be moved around and scaled up (or down) on the fly without affecting the end user).

In some embodiments, the exemplary inventive computer-based systems/platforms, the exemplary inventive computer-based devices, and/or the exemplary inventive computer-based components of the present disclosure may be configured to securely store and/or transmit data by utilizing one or more of encryption techniques (e.g., private/public key pair, Triple Data Encryption Standard (3DES), block cipher algorithms (e.g., IDEA, RC2, RC5, CAST and Skipjack), cryptographic hash algorithms (e.g., MD5, RIPEMD-160, RTR0, SHA-1, SHA-2, Tiger (TTH), WHIRLPOOL, RNGs).

The aforementioned examples are, of course, illustrative and not restrictive.

As used herein, the term “user” shall have a meaning of at least one user. In some embodiments, the terms “user”, “subscriber” “consumer” or “customer” should be understood to refer to a user of an application or applications as described herein and/or a consumer of data supplied by a data provider. By way of example, and not limitation, the terms “user” or “subscriber” can refer to a person who receives data provided by the data or service provider over the Internet in a browser session, or can refer to an automated software application which receives the data and stores or processes the data.

In some embodiments, a method may include providing a bioreactor arrangement including:
a bioreactor chamber for growing a cell culture that may include a plurality of cells or microorganisms in a liquid medium,
a controller,
a plurality of sensors coupled to the bioreactor chamber,
a plurality of control devices for varying a gas flow, a fluid flow, or both in the bioreactor chamber, and
a control circuitry may be configured for receiving control circuitry commands from the controller to control the plurality of control devices;
   where the plurality of sensors may be configured to measure a plurality of sensor parameters in the liquid medium that may include:
   a dissolved oxygen (DO) level,
   a glucose level, and
   a lactate level;
receiving, by the controller through an input device, a desired cell growth configuration;
   where the desired cell growth configuration may include at least one desired value range, at least one setpoint value, or any combination thereof for each of the plurality of sensor parameters measured by the plurality of sensors at predefined time intervals for each of a plurality of cell culture growth parameters during the growth of the cell culture;
receiving, by the controller, at the predefined time intervals, sensor data from each of the plurality of sensors;
inputting, by the controller, the desired cell growth configuration and the sensor data from each of the plurality of sensors, into at least one cell culture control machine learning model;
   where the at least one cell culture control machine learning model may be trained using datasets based at least in part on time-dependent correlations between the plurality of sensor parameters and a plurality of cell culture growth parameters; and
performing, by the controller, based on output data from the at least one cell culture control machine learning model, at least one of:
   transmitting at least one control circuitry command to display on a display, at least one cell culture parameter prediction from the plurality of cell culture growth parameters,
   transmitting at least one control circuitry command to control a gas control device for varying a gas level in the bioreactor chamber,
   transmitting at least one control circuitry command to control a device for controlling a liquid flow of a nutrient fluid into the bioreactor chamber, or
   transmitting at least one control circuitry command to open a device coupled to the bioreactor chamber for removing waste products from the liquid medium.

In some embodiments, the at least one cell culture control machine learning model may be further trained to predict the plurality of cell culture growth parameters of the cell culture based at least on part on at least one correlation between at least one change in at least one sensor parameter value from the plurality of sensor parameters, at least one change in sensor parameter slope from the plurality of sensor parameters at different time points.

In some embodiments, the plurality of sensors may be configured to measure a plurality of sensor parameters in the liquid medium that may include: a pH level, a temperature level, and a pressure.

In some embodiments, the inputting of the sensor data into at least one cell culture control machine learning model may include inputting image data of at least one image of the cell culture in the liquid medium from an imaging camera.

In some embodiments, the inputting of the image data into at least one cell culture control machine learning model may include inputting image data of at least one image of the plurality of cells or microorganisms in the liquid medium received from an imaging camera coupled to a microscope imaging the cell culture.

In some embodiments, the method may include determining, by the controller, a cell count of the plurality of cells or microorganisms in the liquid medium using the image data of the at least one image received from the imaging camera coupled to a microscope imaging the cell culture.

In some embodiments, the at least one cell culture prediction may be selected from the group consisting of:
a prediction of an activation state by a number or percentage of activated cells,
a prediction of cell number,
a prediction of proliferation or differentiation state of cells,
a prediction of cell differentiation,
a prediction of an activated phenotype,
a prediction of a non-activated phenotype,
a prediction of populations, subpopulations, or both within the cell culture that act differently metabolically due to an activation stimulation, a heat shock stimulation, a stress stimulation, and hypoxia,
a prediction of bacterial or a fungal contamination,
a prediction whether T cells react to a target and kill the target,
a prediction when the plurality of cells change and intervention is performed to reactivate or move the plurality of cells to a different media,
a prediction of food contamination,
a prediction of quality control (QC) testing results,
a prediction of an identification of cancer cells in the cell culture due to metabolic consumption,
a prediction of an identification activity of drugs due to changes in cell culture consumption, and
a prediction of drug resistance, drug activity, or both on cancer cells based on a change in metabolism after adding an anti-cancer drug.

In some embodiments, the bioreactor chamber may be partitioned by a perforated barrier into a cone sub-chamber and a media sub-chamber; where the cone sub-chamber may include the plurality of cells; and where the receiving of the sensor data from each of the plurality of sensors may include receiving sensor data from a first set of sensors from the plurality of sensors coupled to the cone sub-chamber and a second set of sensors from the plurality of sensors coupled to the media sub-chamber.

In some embodiments, the receiving the desired cell growth configuration may include receiving additional parameters including:
cell definitions,
cell types,
cell phenotypes at expected sampling times,
conditions for aborting a cell growth run,
differentiation state, and
times to perform at least one action independent of the output data from the at least one cell culture control machine learning model.

In some embodiments, the method may further include generating, by the controller, new datasets using collected data from a plurality of cell sample runs from a respective plurality of subjects; and retraining, by the controller, the at least one cell culture control machine learning model using the new datasets.

A bioreactor system may include:
a bioreactor chamber for growing a cell culture comprising a plurality of cells or microorganisms in a liquid medium;
a controller;
a reservoir chamber for holding a nutrient fluid including at least one nutrient for the plurality of cells or microorganisms;
a plurality of sensors coupled to the bioreactor chamber,
a plurality of control devices to vary a gas flow, a fluid flow, or both in the bioreactor chamber, and
a control circuitry configured to receive control circuitry commands from the controller to control the plurality of control devices;
   wherein the plurality of sensors may be configured to measure a plurality of sensor parameters in the liquid medium that may include:
   a dissolved oxygen (DO) level,
   a glucose level, and
   a lactate level;
wherein the controller may be configured to execute computer code that causes the controller to:
   receive, through an input device, a desired cell growth configuration;
      where the desired cell growth configuration may include at least one desired value range, at least one setpoint value, or any combination thereof for each of the plurality of sensor parameters measured by the plurality of sensors at predefined time intervals for each of a plurality of cell culture growth parameters during the growth of the cell culture;
   receive at the predefined time intervals, sensor data from each of the plurality of sensors;
   input the desired cell growth configuration and the sensor data from each of the plurality of sensors, into a cell culture control machine learning model;
      where the at least one cell culture control machine learning model may be trained using datasets based at least in part on time-dependent correlations between the plurality of sensor parameters and a plurality of cell culture growth parameters; and
   perform based on output data from the cell culture control machine learning model at least one of:
      transmit at least one control circuitry command to display at least one cell culture parameter prediction from the plurality of cell culture growth parameters on a display,
      transmit at least one control circuitry command to control a gas control device for varying a gas level in the bioreactor chamber,
      transmit at least one control circuitry command to control a device for controlling a liquid flow of a nutrient fluid into the bioreactor chamber, or
      transmit at least one control circuitry command to open a device coupled to the bioreactor chamber for removing waste products from the liquid medium.

In some embodiments, the at least one cell culture control machine learning model may be further trained to predict the plurality of cell culture growth parameters of the cell culture based at least on part on at least one correlation between at least one change in at least one sensor parameter value from the plurality of sensor parameters, at least one change in sensor parameter slope from the plurality of sensor parameters at different time points.

In some embodiments, the plurality of sensors may be configured to measure a plurality of sensor parameters in the liquid medium that may include:
a pH level,
a temperature level, and
a pressure.

In some embodiments, the controller may be configured to input the sensor data into at least one cell culture control machine learning model by inputting image data of at least one image of the cell culture in the liquid medium from an imaging camera.

In some embodiments, the controller may be configured to input the image data into at least one cell culture control machine learning model by inputting image data of at least one image of the plurality of cells or microorganisms in the liquid medium received from an imaging camera coupled to a microscope imaging the cell culture.

In some embodiments, the controller may be further configured to determine a cell count of the plurality of cells or microorganisms in the liquid medium using the image data of the at least one image received from the imaging camera coupled to a microscope imaging the cell culture.

In some embodiments, the at least one cell culture prediction may be selected from the group consisting of:
a prediction of an activation state by a number or percentage of activated cells,
a prediction of cell number,
a prediction of proliferation or differentiation state of cells,
a prediction of cell differentiation,
a prediction of an activated phenotype,
a prediction of a non-activated phenotype,
a prediction of populations, subpopulations, or both within the cell culture that act differently metabolically due to an activation stimulation, a heat shock stimulation, a stress stimulation, and hypoxia,
a prediction of bacterial or a fungal contamination,
a prediction whether T cells react to a target and kill the target,
a prediction when the plurality of cells change and intervention is performed to reactivate or move the plurality of cells to a different media,
a prediction of food contamination,
a prediction of quality control (QC) testing results,
a prediction of an identification of cancer cells in the cell culture due to metabolic consumption,
a prediction of an identification activity of drugs due to changes in cell culture consumption, and
a prediction of drug resistance, drug activity, or both on cancer cells based on a change in metabolism after adding an anti-cancer drug.

In some embodiments, the bioreactor chamber may be partitioned by a perforated barrier into a cone sub-chamber and a media sub-chamber; where the cone sub-chamber may include the plurality of cells; and where the controller may be configured to receive the sensor data from each of the plurality of sensors by receiving sensor data from a first set of sensors from the plurality of sensors coupled to the cone sub-chamber and a second set of sensors from the plurality of sensors coupled to the media sub-chamber.

In some embodiments, the controller may be configured to receive the desired cell growth configuration by receiving additional parameters comprising:
cell definitions,
cell types,
cell phenotypes at expected sampling times,
conditions for aborting a cell growth run,
differentiation state, and
times to perform at least one action independent of the output data from the at least one cell culture control machine learning model.

In some embodiments, the controller may be further configured to:
generate new datasets using collected data from a plurality of cell sample runs from a respective plurality of subjects; and
retrain the at least one cell culture control machine learning model using the new datasets.

Publications cited throughout this document are hereby incorporated by reference in their entirety. While one or more embodiments of the present disclosure have been described, it is understood that these embodiments are illustrative only, and not restrictive, and that many modifications may become apparent to those of ordinary skill in the art, including that various embodiments of the inventive methodologies, the inventive systems/platforms, and the inventive devices described herein can be utilized in any combination with each other. Further still, the various steps may be carried out in any desired order (and any desired steps may be added and/or any desired steps may be eliminated).

1. A method, comprising:
providing a bioreactor arrangement comprising:
a bioreactor chamber for growing a cell culture comprising a plurality of cells or microorganisms in a liquid medium,
a controller,
a plurality of sensors coupled to the bioreactor chamber,
a plurality of control devices for varying a gas flow, a fluid flow, or both in the bioreactor chamber, and
a control circuitry configured for receiving control circuitry commands from the controller to control the plurality of control devices;
wherein the plurality of sensors is configured to measure a plurality of sensor parameters in the liquid medium that comprises:
a dissolved oxygen (DO) level,
a glucose level, and
a lactate level;
receiving, by the controller through an input device, a desired cell growth configuration;
wherein the desired cell growth configuration comprises at least one desired value range, at least one setpoint value, or any combination thereof for each of the plurality of sensor parameters measured by the plurality of sensors at predefined time intervals for each of a plurality of cell culture growth parameters during the growth of the cell culture;
receiving, by the controller, at the predefined time intervals, sensor data from each of the plurality of sensors;
inputting, by the controller, the desired cell growth configuration and the sensor data from each of the plurality of sensors, into at least one cell culture control machine learning model;
wherein the at least one cell culture control machine learning model is trained using datasets based at least in part on time-dependent correlations between the plurality of sensor parameters and a plurality of cell culture growth parameters; and
performing, by the controller, based on output data from the at least one cell culture control machine learning model, at least one of:
transmitting at least one control circuitry command to display on a display, at least one cell culture parameter prediction from the plurality of cell culture growth parameters,
transmitting at least one control circuitry command to control a gas control device for varying a gas level in the bioreactor chamber,
transmitting at least one control circuitry command to control a device for controlling a liquid flow of a nutrient fluid into the bioreactor chamber, or
transmitting at least one control circuitry command to open a device coupled to the bioreactor chamber for removing waste products from the liquid medium.

2. The method according to claim 1, wherein the at least one cell culture control machine learning model is further trained to predict the plurality of cell culture growth parameters of the cell culture based at least on part on at least one correlation between at least one change in at least one sensor parameter value from the plurality of sensor parameters, at least one change in sensor parameter slope from the plurality of sensor parameters at different time points.

3. The method according to claim 1, wherein the plurality of sensors is configured to measure a plurality of sensor parameters in the liquid medium that comprises:
a pH level,
a temperature level, and
a pressure.

4. The method according to claim 1, wherein the inputting of the sensor data into at least one cell culture control machine learning model comprises inputting image data of at least one image of the cell culture in the liquid medium from an imaging camera.

5. The method according to claim 4, wherein the inputting of the image data into at least one cell culture control machine learning model comprises inputting image data of at least one image of the plurality of cells or microorganisms in the liquid medium received from an imaging camera coupled to a microscope imaging the cell culture.

6. The method according to claim 5, further comprising determining, by the controller, a cell count of the plurality of cells or microorganisms in the liquid medium using the image data of the at least one image received from the imaging camera coupled to a microscope imaging the cell culture.

7. The method according to claim 1, wherein the at least one cell culture prediction is selected from the group consisting of:
a prediction of an activation state by a number or percentage of activated cells,
a prediction of cell number,
a prediction of proliferation or differentiation state of cells,
a prediction of cell differentiation,
a prediction of an activated phenotype,
a prediction of a non-activated phenotype,
a prediction of populations, subpopulations, or both within the cell culture that act differently metabolically due to an activation stimulation, a heat shock stimulation, a stress stimulation, and hypoxia,
a prediction of bacterial or a fungal contamination,
a prediction whether T cells react to a target and kill the target,
a prediction when the plurality of cells change and intervention is performed to reactivate or move the plurality of cells to a different media,
a prediction of food contamination,
a prediction of quality control (QC) testing results,
a prediction of an identification of cancer cells in the cell culture due to metabolic consumption,
a prediction of an identification activity of drugs due to changes in cell culture consumption, and
a prediction of drug resistance, drug activity, or both on cancer cells based on a change in metabolism after adding an anti-cancer drug.

8. The method according to claim 1, wherein the bioreactor chamber is partitioned by a perforated barrier into a cone sub-chamber and a media sub-chamber;
wherein the cone sub-chamber comprises the plurality of cells; and
wherein the receiving of the sensor data from each of the plurality of sensors comprises receiving sensor data from a first set of sensors from the plurality of sensors coupled to the cone sub-chamber and a second set of sensors from the plurality of sensors coupled to the media sub-chamber.

9. The method according to claim 1, wherein the receiving the desired cell growth configuration comprises receiving additional parameters comprising:
cell definitions,
cell types,
cell phenotypes at expected sampling times,
conditions for aborting a cell growth run,
differentiation state, and
times to perform at least one action independent of the output data from the at least one cell culture control machine learning model.

10. The method according to claim 1, further comprising:
generating, by the controller, new datasets using collected data from a plurality of cell sample runs from a respective plurality of subjects; and
retraining, by the controller, the at least one cell culture control machine learning model using the new datasets.

11. A bioreactor system, comprising:
a bioreactor chamber for growing a cell culture comprising a plurality of cells or microorganisms in a liquid medium;
a controller;
a reservoir chamber for holding a nutrient fluid comprising at least one nutrient for the plurality of cells or microorganisms;
a plurality of sensors coupled to the bioreactor chamber,
a plurality of control devices to vary a gas flow, a fluid flow, or both in the bioreactor chamber, and
a control circuitry configured to receive control circuitry commands from the controller to control the plurality of control devices;
wherein the plurality of sensors is configured to measure a plurality of sensor parameters in the liquid medium that comprises:
a dissolved oxygen (DO) level,
a glucose level, and
a lactate level;
wherein the controller is configured to execute computer code that causes the controller to:
receive, through an input device, a desired cell growth configuration;
wherein the desired cell growth configuration comprises at least one desired value range, at least one setpoint value, or any combination thereof for each of the plurality of sensor parameters measured by the plurality of sensors at predefined time intervals for each of a plurality of cell culture growth parameters during the growth of the cell culture;
receive at the predefined time intervals, sensor data from each of the plurality of sensors;
input the desired cell growth configuration and the sensor data from each of the plurality of sensors, into a cell culture control machine learning model;
wherein the at least one cell culture control machine learning model is trained using datasets based at least in part on time-dependent correlations between the plurality of sensor parameters and a plurality of cell culture growth parameters; and
perform based on output data from the cell culture control machine learning model at least one of:
transmit at least one control circuitry command to display at least one cell culture parameter prediction from the plurality of cell culture growth parameters on a display,
transmit at least one control circuitry command to control a gas control device for varying a gas level in the bioreactor chamber,
transmit at least one control circuitry command to control a device for controlling a liquid flow of a nutrient fluid into the bioreactor chamber, or
transmit at least one control circuitry command to open a device coupled to the bioreactor chamber for removing waste products from the liquid medium.

12. The bioreactor system according to claim 11, wherein the at least one cell culture control machine learning model is further trained to predict the plurality of cell culture growth parameters of the cell culture based at least on part on at least one correlation between at least one change in at least one sensor parameter value from the plurality of sensor parameters, at least one change in sensor parameter slope from the plurality of sensor parameters at different time points.

13. The bioreactor system according to claim 11, wherein the plurality of sensors is configured to measure a plurality of sensor parameters in the liquid medium that comprises:
a pH level,
a temperature level, and
a pressure.

14. The bioreactor system according to claim 11, wherein the controller is configured to input the sensor data into at least one cell culture control machine learning model by inputting image data of at least one image of the cell culture in the liquid medium from an imaging camera.

15. The bioreactor system according to claim 14, wherein the controller is configured to input the image data into at least one cell culture control machine learning model by inputting image data of at least one image of the plurality of cells or microorganisms in the liquid medium received from an imaging camera coupled to a microscope imaging the cell culture.

16. The bioreactor system according to claim 15, wherein the controller is further configured to determine a cell count of the plurality of cells or microorganisms in the liquid medium using the image data of the at least one image received from the imaging camera coupled to a microscope imaging the cell culture.

17. The bioreactor system according to claim 11, wherein the at least one cell culture prediction is selected from the group consisting of:
a prediction of an activation state by a number or percentage of activated cells,
a prediction of cell number,
a prediction of proliferation or differentiation state of cells,
a prediction of cell differentiation,
a prediction of an activated phenotype,
a prediction of a non-activated phenotype,
a prediction of populations, subpopulations, or both within the cell culture that act differently metabolically due to an activation stimulation, a heat shock stimulation, a stress stimulation, and hypoxia,
a prediction of bacterial or a fungal contamination,
a prediction whether T cells react to a target and kill the target,
a prediction when the plurality of cells change and intervention is performed to reactivate or move the plurality of cells to a different media,
a prediction of food contamination,
a prediction of quality control (QC) testing results,
a prediction of an identification of cancer cells in the cell culture due to metabolic consumption,
a prediction of an identification activity of drugs due to changes in cell culture consumption, and
a prediction of drug resistance, drug activity, or both on cancer cells based on a change in metabolism after adding an anti-cancer drug.

18. The bioreactor system according to claim 11, wherein the bioreactor chamber is partitioned by a perforated barrier into a cone sub-chamber and a media sub-chamber;
wherein the cone sub-chamber comprises the plurality of cells; and
wherein the controller is configured to receive the sensor data from each of the plurality of sensors by receiving sensor data from a first set of sensors from the plurality of sensors coupled to the cone sub-chamber and a second set of sensors from the plurality of sensors coupled to the media sub-chamber.

19. The bioreactor system according to claim 11, wherein the controller is configured to receive the desired cell growth configuration by receiving additional parameters comprising:
cell definitions,
cell types,
cell phenotypes at expected sampling times,
conditions for aborting a cell growth run,
differentiation state, and
times to perform at least one action independent of the output data from the at least one cell culture control machine learning model.

20. The bioreactor system according to claim 11, wherein the controller is further configured to:
generate new datasets using collected data from a plurality of cell sample runs from a respective plurality of subjects; and
retrain the at least one cell culture control machine learning model using the new datasets.
